(19) United States
(12) Patent Application Publication
(10) Pub. No.: US 2026/0209243 A1
(43) Pub. Date: Jul. 23, 2026

(54) A BIOORTHOGONAL CYCLOADDITION REACTION AND USE THEREOF

(71) Applicant: NANJING UNIVERSITY, Jiangsu (CN)

(72) Inventors: Yan ZHANG, Jiangsu (CN); Huan WANG, Jiangsu (CN); Jinbo LI, Jiangsu (CN)

(73) Assignee: NANJING UNIVERSITY, Jiangsu (CN)

(21) Appl. No.: 19/141,379

(22) PCT Filed: Dec. 19, 2023
(86) PCT No.: PCT/CN2023/139889

§ 371 Date: Jun. 20, 2025

(30) Foreign Application Priority Data
Dec. 20, 2022 (WO) PCT/CN2022/140215

(51) Int. Cl. C07D 493/04; A61K 47/54; A61K 47/55; A61K 49/00

(52) U.S. Cl. CPC C07D 493/04; A61K 47/545; A61K 47/55; A61K 49/0052

(57) ABSTRACT

The present disclosure provides a bioorthogonal cycloaddition reaction, as well as the use in assembly of TAC-type molecules and use in preparation of bioconjugates.

### TECHNICAL FIELD

The present application relates to the field of biomedicine, in particular to a bioorthogonal cycloaddition reaction and use thereof.

### BACKGROUND OF THE INVENTION

Expanding the bioorthogonal tool-box with mutually orthogonal bioorthogonal cycloadditions is of great importance. However, only a few examples of triple orthogonal labelling of biomolecules may have been reported to date, effective bioorthogonal cycloadditions with high accessibility and stability are still highly desired.

### SUMMARY OF THE INVENTION

The present application provides a method of performing a coupling reaction, comprising providing a first structure comprising a dione group or derivative thereof and a second structure capable of providing anionic furan-2-olate or derivative thereof

The present application provides a combination, comprising a first structure comprising a dione group or derivative thereof and a second structure capable of providing anionic furan-2-olate or derivative thereof.

The present application provides a method for preparing said combination, comprising linking a first structure with a dione group or derivative thereof and linking a second structure with a group capable of providing anionic furan-2-olate or derivative thereof.

The present application provides a conjugate, said conjugate is prepared using method of the present application.

The present application provides a kit, comprising combination of the present application, and/or conjugate of the present application.

The present application provides a method for promoting cycloaddition reaction, comprising regulating electrostatic interactions between reactants.

The present application provides a combination, comprising reactants with regulated electrostatic interaction.

The present application provides a method for performing two, three or more bioorthogonal reactions, wherein one of the bioorthogonal reaction comprises dione-based cycloaddition reaction.

The present application provides a cell and/or cell set with structure capable of performing two, three or more bioorthogonal reactions, wherein one of the bioorthogonal reaction comprises dione-based cycloaddition reaction.

The present application provides a protein and/or protein set with structure capable of performing two, three or more bioorthogonal reactions, wherein one of the bioorthogonal reaction comprises dione-based cycloaddition reaction.

The present application provides a kit comprising cell and/or cell set of the present application, and/or protein and/or protein set of the present application.

The present application provides a method for preparing (targeting chimera) TAC-type molecule, said method comprises providing dione-based cycloaddition reaction.

The present application provides a TAC-type molecule prepared using method of the present application.

The present application provides a method for preparing bi-functional bioconjugate and/or cyclic peptide, said method comprises providing dione-based cycloaddition reaction.

The present application provides a bi-functional bioconjugate and/or cyclic peptide prepared using method of the present application.

The present application provides a method for identification a target, comprising adding a tag on a natural product comprising a dione group or derivative thereof, wherein the tag comprises an anionic furan-2-olate or derivative thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

As shown in FIG. 1. The present strategy the application established for photo trigger-free, water-promoted cycloaddition between phenanthrene-9,10-dione and furan-2(3H)-one derivatives.

As shown in FIGS. 2A-2C. The cycloaddition kinetics of α-AL with different o-diones in pH 7.4 PBS buffer or in aqueous solution with indicated pH.

As shown in FIGS. 3A-3D. DFC reaction in PBS and on proteins. (A) The DFC reaction of oD1 with FuA in pH 7.4 PBS buffer; (B) Time-dependent labelling of 2 mg/mL BSA-FuA with 100 nM oD1-TAMRA for time indicated. Fluorescence signals were acquired using Cy3 channel. Coomassie staining was used to assess BSA loading; (C) Schematic illustration on the single-site modification of the protein oD1 (C156)-SNAP-GFP through the DFC reaction. SNAP-GFP (2 mg/mL) in PBS (pH=7.4) was mixed with 1.5 equivalents of BG-PEG₄-oD1 and was incubated at room temperature for 20 min to give oD1 (C156)-SNAP-GFP. Then 1.5 equivalents of FuA-TAMRA was added into the above solution and incubated for 20 min at room temperature; (D) LC-MS/MS characterization of the fragment containing the oD1-modified at the C156 residue in SNAP-GFP fusion protein before (above) and after (below) the DFC reaction.

As shown in FIGS. 4A-4D. Fluorescence labelling of live cells by the DFC reaction. (A) Schematic illustration of random cell membrane protein labelling by DFC reaction; (B) Confocal image of cell membrane protein labelling. SKOV3 cells were firstly incubated with FuA-PEG₄-NHS (100 μM in PBS) for 30 min and then with oD1-TAMRA (20 μM in PBS) for 10 min at room temperature; (C) Schematic illustration of cell membrane glycan labelling by DFC reaction; (D) confocal image of cell membrane glycan labelling. CHO cells were firstly incubated with GDP-Fucose-oD1 (100 μM in HBSS buffer) for 20 min at 4° C. and then with FuA-TAMRA (20 UM in PBS) for 10 min at room temperature. Scale bar: 50 μm.

As shown in FIG. 5. Schematic illustration and SDS-PAGE analysis result of orthogonal labelling of three different proteins based on DFC, SPAAC and IEDDA reactions. For each reaction mixture, the final concentrations of BSA-TCO, OVA-oD1, LYSO-Azwere 0.5 mg/mL, 0.75 mg/mL, 2.5 mg/mL respectively and the final concentrations of Tz-Cy5, FuA-TAMRA, DBCO-FITC were all about 200 μM. The mixtures were incubated at room temperature for 5 min.

As shown in FIGS. 6A-6D. Orthogonal labelling of three different cells based on DFC, SPAAC and IEDDA reactions monitored with flow cytometry. (A) Scheme illustration of the labelling strategy; (B) Three different cell groups distinguished by EGFR and HER2 antibody; (C) Fluorescence signal analysis of the three kinds of cells. The mixture of A549, SKOV3 and N87 cells or the mixture of A549-TCO, SKOV3-oD1 and N87-DBCO cells were incubated with the PBS solution containing 2 μM Tz-Cy5, 20 μM FuA-TAMRA and 20 μM Az-FITC for 5~10 min at room temperature; (D) Fluorescence intensity multiples of labelled cells with respect to control cell.

As shown in FIG. 7. The application provides the first metal-, strain- and photo trigger-free, anionic cycloaddend promoted bioorthogonal cycloaddition reaction between phenanthrene-9,10-dione and furan-2(3H)-one derivatives was reported. Mutually orthogonal labelling of different proteins and cells were realized using three bioorthogonal reactions including DFC, SPAAC and IEDDA.

As shown in FIG. 8A-8B. Measurement of second order rate constants of (A) reactions of o-dione (25 μM) with furan-2(3H)-one (250 μM) in 1×PBS (pH=7.4) and (B) reactions of oD1 (25 μM) with α-AL (250 μM) in different pH buffer. (When pH=5, 6; oD1 is 250 μM and α-AL is 2.5 mM) by plotting ln[1/(1−x)] versus time of o-dione with furan-2(3H)-one reaction (x represents the percent conversion of o-dione).

As shown in FIG. 9A-9D. DFC-based BSA protein labelling and characterization. (A) Schematic illustration of the protocol to prepare the modified protein BSA-FuA and to label the protein through DFC reaction. 2 mg/mL BSA was mixed with 200 equivalents of FuA-NHS ester and shaken at room temperature for 4 h. 2 mg/mL BSA-FuA was mixed with 50 equivalents of PQ and shaken at room temperature for 1 h; (B) LC-MS/MS characterization of the fragments containing the FuA-modified lysine residue in BSA protein before and after the DFC reaction. One of the FuA-modified fragments is showed; (C) dose-dependent labelling of BSA-FuA on samples of BSA or BSA-FuA mixed with the indicated amount of oD1-TAMRA at room temperature for 30 min.

As shown in FIG. 10. Native-PAGE detection of single-site modification of SNAP-GFP using the DFC reaction. The 2 mg/mL PBS solution of SNAP-GFP was mixed with 1.5 equivalent BG-PEG₄-oD1 and incubated at room temperature for 20 min. Then 1.5 equivalent FuA-TAMRA was added into the above solution and further incubated for 0-5 min.

As shown in FIG. 11A-11B. Illustration of cells labelling with FuA or OD1. (A) Schematic illustration of random cell membrane protein labelling using FuA-NHS. SKOV3 cell were incubated with 100 μM FuA-NHS PBS solution at room temperature for 30 min; (B) Schematic illustration of cell membrane glycan labelling using GDP-Fucose-oD1. CHO cells were incubated with GDP-Fucose-oD1 (100 μM in HBSS buffer) for 20 min at 4° C.

As shown in FIG. 12. Synthetic method of GDP-Fucose-oD1. HRMS (ESI) calcd. for C₃₄H₃₄N₉O₁₈P₂ 918.1497 [M−H]⁻; found 918.1514.

As shown in FIG. 13. Relative cell viability of SKOV3 cells after incubation with FuA or PDO6. SKOV3 cells were incubated with 0-200 μM FuA or PDO6 for 24 h. MTT was used to assess the cell viability. Data are shown as mean±SEM (n=3).

As shown in FIG. 14. Illustration of proteins labelling with TCO, oD1 or Az. 5 mg/mL BSA, 5 mg/mL OVA and 10 mg/mL LYSO were incubated with 50 eq TCO-NHS, 50 eq oD1-PEG₄-NHS and 7 eq Az-PEG₃-NHS respectively for 2 h in PBS at room temperature.

As shown in FIG. 15. Raw-files for the SDS-PAGE images of triple orthogonal protein labelling under different fluorescent imaging channels.

As shown in FIG. 16. Illustration of cells labeling with TCO, oDI or DBCO. A549, SKOV3 and N87 cells were incubated with 50 μM TCO-NHS, 50 μM oD1-PEG₄-NHS and 50 μM DBCO-NHS respectively for 20 min in PBS at room temperature.

As shown in FIG. 17A-17F. Cell images of negative controls for triple bioorthogonal cell labelling. (A) A549 cells were firstly incubated with 50 μM TCO-NHS for 20 min and then with 20 μM FuA-TAMRA for 5 min; (B) A549 cells were firstly incubated with 50 μM TCO-NHS for 20 min and then with 20 μM Az-FITC for 5 min; (C) SKOV3 cells were firstly incubated with 50 μM oD1-PEG₄-NHS for 20 min and then with 2 μM Tz-Cy5 for 5 min; (D) SKOV3 cells were firstly incubated with 50 μM oD1-PEG₄-NHS for 20 min and then with 20 μM Az-FITC for 5 min; (E) N87 cells were firstly incubated with 50 μM DBCO-NHS for 20 min and then with 2 μM Tz-Cy5 for 5 min; (F) N87 cells were firstly incubated with 50 μM DBCO-NHS for 20 min and then with 20 μM FuA-TAMRA for 5 min. All the reactions were performed at room temperature. Scale bar: 20 μm.

As shown in FIG. 18. The application provides in vivo labeling using the DFC reaction.

As shown in FIG. 19. The result shows cells cultured with AL-NHS or controls.

As shown in FIG. 20. The result shows cells labeled with AL were injected subcutaneously into subjects.

As shown in FIG. 21. Using bifunctional chimeras for DFC reaction is also able to generate A-linker-B.

As shown in FIG. 22. Using bifunctional chimeras for DFC reaction inside cells.

As shown in FIG. 23. Using bifunctional chimeras for Antibody-peptide conjugates, antibody-nucleic acid conjugates, peptide-nucleic acid peptides, antibody-oligosaccharide conjugates, peptide-lipid conjugates, antibody-small molecule conjugates, and/or nucleic acid-small molecule conjugates.

As shown in FIG. 24. Using bifunctional chimeras for preparing drug conjugate containing cyclic peptides.

As shown in FIG. 25. An example for preparing drug conjugate containing cyclic peptides.

As shown in FIG. 26. Cyclic-peptide modified with D/F can be conjugated using DFC reaction.

As shown in FIG. 27. Cyclic-peptide modified with F and azide can be orthogonal coupled to two different cyclic-peptides modified with D/Dibenzocyclooctyne respectively.

FIG. 28 illustrates that the DFC reaction route between JQ1 molecule and PQ molecule.

FIG. 29 illustrates that the synthesis route of PQ-N-Ad.

FIG. 30 illustrates that results of DFC reaction between JQ1 molecule and PQ molecule in solution.

FIG. 31 illustrates that results of DFC reaction between JQ1 molecule and PQ molecule in cell.

FIG. 32 illustrates that activity of products obtained from DFC reaction in targeting and degradation.

FIG. 33 illustrates that results of DFC reaction between Rnase-al and PQ molecule.

FIG. 34 illustrates that DFC reaction scheme of Tanshione I and a-Al.

FIG. 35 illustrates that the results after DFC reaction between Tanshione I and a-Al.

FIG. 36 illustrates that DFC reaction scheme of Tanshione IIA and a-Al.

FIG. 37 illustrates that results after DFC reaction between Tanshione IIA and a-Al.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Any suitable substituent may be used in a substituted alkyl group, a substituted aryl group, or a substituted heteroaryl group, including, but not limited to, an alkyl group, a halogen, an aryl group, and a heteroaryl group, as defined hereinabove, Boc-amino group, protected amino acids, nitro group, methoxy group, nitrile group, ester group, heterocycles, biotin.

The term “conjugate”, as used herein, generally refers to any substance formed from the joining together of separate parts. In the conjugate, the separate parts may be joined at one or more active site with each other. Moreover, the separate parts may be covalently or non-covalently associated with, or linked to, each other and exhibit various stoichiometric molar ratios. The conjugate may comprise peptides, polypeptides, proteins, prodrugs which are metabolized to an active agent in vivo, polymers, nucleic acid molecules, small molecules, binding agents, mimetic agents, synthetic drugs, inorganic molecules, organic molecules and radioisotopes.

The term “molecule of interest (MOI)”, as used herein, generally refers to a molecule with a desired characteristic. The desired characteristic may be a physical characteristic or a chemical characteristic, for example, reactive activity, stability, solubility, binding activity, inhibiting activity, toxicity or degradability. A MOI may comprise any substances possessing a desired biological activity and/or a reactive functional group that may be used to incorporate a drug into the protein conjugate of the disclosure. For example, a MOI may comprise an active substance. For example, the active substance may be a therapeutical agent, a diagnosis agent, a pharmacological agent and/or a biological agent, e.g., a cytotoxin, a cytostatic agent, a radioisotope or radionuclide, a metal chelator, an oligonucleotide, an antibiotic, a fluorophore, a biotin tag, a peptide, a protein, or any combination thereof. In some cases, an active substance could be a chemically active substance. For example, a chemically active substance may be a chemically functional moiety that could reacted with another chemically functional moiety to form a covalent bond. For example, a chemically active substance may be able to participate in a ligation reaction. In some cases, an active substance could be an enzymatically active substance that could be reacted with complementary functional moiety to form a covalent bond in the presence of an enzyme.

The term “hydrocarbyl”, as used herein, generally refers to a moiety consisting exclusively of hydrogen and carbon atoms; such a moiety may comprise an aliphatic and/or an aromatic moiety. The moiety may comprise 1, 2, 3, 4, 5, 6, 7, 8, 9, 10, 11, 12, 13, 14, 15, 16, 17, 18, 19, 20, 30, 40, 50 or more carbon atoms. Examples of hydrocarbyl groups include without limitation alkyl such as C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g cyclopropylmethyl); cycloalkyl (e.g. cyclopropyl, cyclobutyl, cyclopentyl or cyclohexyl); aryl (e.g. phenyl, naphthyl or fluorenyl) and the like.

The term “heterohydrocarbyl”, as used herein, generally refers to a hydrocarbyl group that optionally includes one or more heteroatoms. The heteroatoms may be any atom other than C, such as a O, S or N.

The term “alkenyl”, as used herein, generally refers to a straight or branched chain alkyl moiety having 2, 3, 4, 5, 6 or more carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

The term “aryl”, as used herein, generally refers to an aromatic ring system comprising 6, 7, 8, 9, 10, 11, 12, 13, 14, 15, 16 or more ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate.

The terms “comprise(s),” “include(s),” “having,” “has,” “can,” “contain(s),” and variants thereof, as used herein, generally are intended to be open--ended transitional phrases, terms, or words that do not preclude the possibility of additional acts or structures. The singular forms “a,” “and” and “the” include plural references.

### The Bioorthogonal Cycloaddition Reaction

In one aspect, the present application provides a method of performing a coupling reaction, comprising providing a first structure comprising a dione group or derivative thereof and a second structure capable of providing anionic furan-2-olate or derivative thereof.

For example, said second structure may comprise a furan-2(3H)-one or derivative thereof.

For example, said furan-2(3H)-one is optionally substituted; when said furan-2(3H)-one is substituted, said substitution is selected form optional substituents. Any suitable substituent may be used in a substituted alkyl group, a substituted aryl group, or a substituted heteroaryl group, including, but not limited to, an alkyl group, a halogen, an aryl group, and a heteroaryl group, as defined hereinabove, Boc-amino group, protected amino acids, nitro group, methoxy group, nitrile group, ester group, heterocycles, biotin.

For example, said second structure further may comprise small molecule, peptide, nucleic acids, peptide nucleic acid (PNA), oligosaccharides, glycans, and/or lipid. For example, said second structure further may comprise therapeutic drug, toxin, and/or detectable substance. For example, said second structure further may comprise antibody, fluorescent protein, membrane bound protein and/or glycoprotein.

For example, said first structure comprising an o-dione group or derivative thereof.

For example, said first structure comprising a phenanthrene-9,10-dione or derivative thereof. For example, said dione group is optionally substituted; when said dione group is substituted, said substitution is selected form optional substituents. Any suitable substituent may be used in a substituted alkyl group, a substituted aryl group, or a substituted heteroaryl group, including, but not limited to, an alkyl group, a halogen, an aryl group, and a heteroaryl group, as defined hereinabove, Boc-amino group, protected amino acids, nitro group, methoxy group, nitrile group, ester group, heterocycles, biotin.

For example, said first structure further may comprise small molecule, peptide, nucleic acids, peptide nucleic acid (PNA), oligosaccharides, glycans, and/or lipid. For example, said first structure further may comprise therapeutic drug, toxin, and/or detectable substance. For example, said first structure further may comprise antibody, fluorescent protein, membrane bound protein and/or glycoprotein.

For example, said method further may comprise adding said dione group to said first structure and/or adding said group capable of providing anionic furan-2-olate or derivative thereof to said second structure.

For example, said method is performed at 4° C. to room temperature and/or at pH 5 to 9. For example, said method is performed at about 4° C., about 5° C., about 10° C., about 20° C., about 25° C., about 30° C., about 35° C., about 40° C., about 50° C., or about 100° C. For example, said method is performed at pH 1, 2, 3, 4, 5, 6, 7, 7.1, 7.2, 7.3, 7.4, 7.5, 8, 9, 10, 11, 12, 13 or 14.

For example, said method is performed for 30 minutes or less. For example, said method is performed for 1 second, 2 seconds, 3 seconds, 4 seconds, 5 seconds, 10 seconds, 15 seconds, 20 seconds, 25 seconds, 30 seconds, 35 seconds, 40 seconds, 50 seconds, 60 seconds, or 1 minute, 2 minutes, 3 minutes, 4 minutes, 5 minutes, 10 minutes, 15 minutes, 20 minutes, 25 minutes, 30 minutes, 40 minutes, 50 minutes, 100 minutes, or 1 hour, 2 hours, 3 hours, 4 hours, 5 hours, 10 hours, 15 hours, 20 hours, 25 hours, 30 hours, 40 hours, 50 hours, or 100 hours.

For example, said method is performed by combining said first structure and said second structure in about 1:100 to about 100:1 ratio. For example, said method is performed by combining said first structure and said second structure in about 1:10000, 1:1000, 1:100, 1:10, 1:5, 1:2, 1:1, 2:1, 5:1, 10:1, 100:1, 1000:1, 10000:1, 100000:1 ratio or equivalents.

For example, said method is performed in vitro and/or in vivo. For example, said method is performed in subject's body.

The present application provides a combination, comprising a first structure comprising a dione group or derivative thereof and a second structure capable of providing anionic furan-2-olate or derivative thereof.

The present application provides a method for preparing said combination, comprising linking a first structure with a dione group or derivative thereof and linking a second structure with a group capable of providing anionic furan-2-olate or derivative thereof.

The present application provides a conjugate, said conjugate is prepared using method of the present application.

The present application provides a kit, comprising combination of the present application, and/or conjugate of the present application.

The present application provides a method for promoting cycloaddition reaction, comprising regulating electrostatic interactions between reactants.

For example, comprising enhancing electrostatic interactions between transition-state structures of said reactants. For example, comprising promoting electron transfer process from vinyl ether to the excited o-dione at ππ* state. For example, comprising providing electron-rich structure. For example, comprising providing electron-rich C═C bond.

For example, comprising providing group capable of providing anionic furan-2-olate or derivative thereof. For example, comprising providing furan-2(3H)-one or derivative thereof.

The present application provides a combination, comprising reactants with regulated electrostatic interaction.

The present application provides a method for performing two, three or more bioorthogonal reactions, wherein one of the bioorthogonal reaction may comprise dione-based cycloaddition reaction.

For example, wherein said dione-based cycloaddition reaction may comprise method of the present application.

For example, wherein two, three or more of said bioorthogonal reactions are performed simultaneously in one batch and/or without cross-talking. For example, wherein 2, 3, 4, 5, 10, 100 or more of said bioorthogonal reactions are performed simultaneously in one batch and/or without cross-talking.

For example, wherein two, three or more of said bioorthogonal reactions are performed simultaneously for mutually orthogonal labelling of three types of proteins or three groups of living cells. For example, wherein 2, 3, 4, 5, 10, 100 or more of said bioorthogonal reactions are performed simultaneously for mutually orthogonal labelling of 2, 3, 4, 5, 10, 100 or more types of proteins or 2, 3, 4, 5, 10, 100 or more groups of living cells.

For example, said two, three or more bioorthogonal reactions are configured for labelling of two or more proteins and/or live cells. For example, said 2, 3, 4, 5, 10, 100 or more bioorthogonal reactions are configured for labelling of 2, 3, 4, 5, 10, 100 or more proteins and/or live cells.

For example, wherein one of the bioorthogonal reaction may comprise cycloaddition. For example, wherein one of the bioorthogonal reaction may comprise Cu-catalyzed azide-alkyne click reaction (CuAAC), the strain-promoted azide-alkyne click reaction (SPAAC) and/or the inverse-electron-demand Diels-Alder reaction (IEDDA).

The present application provides a cell and/or cell set with structure capable of performing two, three or more bioorthogonal reactions, wherein one of the bioorthogonal reaction may comprise dione-based cycloaddition reaction.

The present application provides a protein and/or protein set with structure capable of performing two, three or more bioorthogonal reactions, wherein one of the bioorthogonal reaction may comprise dione-based cycloaddition reaction.

The present application provides a kit comprising cell and/or cell set of the present application, and/or protein and/or protein set of the present application.

The development of new bioorthogonal reactions with mutual orthogonality to classic bioorthogonal reactions such as the strain-promoted azide-alkyne click reaction and the inverse-electron-demand Diels-Alder reaction is of great importance in providing chemical tools for multiplex labelling of live cells. Here the application provides the first anionic cycloaddend-promoted bioorthogonal cycloaddition reaction between phenanthrene-9,10-dione and furan-2(3H)-one derivatives, where the high polarity of water is exploited to stabilize the highly electron-rich anionic cycloaddend. The reaction is metal- and strain-free, which proceeds rapidly in aqueous solution and on live cells with a second-order rate constants up to 119 M⁻¹ s⁻¹. The combined utilization of this reaction together with the two other widely used bioorthogonal reactions allows for mutually orthogonal labelling of three types of proteins or three groups of living cells in one batch without cross-talking. Such results highlight the great potential for multiplex labelling of different biomolecules in live cells.

Bioorthogonal chemical tools have found diverse biological applications and greatly accelerated the pace of innovations in chemical biology. Among the well-established bioorthogonal or biocompatible reactions, cycloadditions such as the Cu-catalyzed azide-alkyne click reaction (CuAAC), the strain-promoted azide-alkyne click reaction (SPAAC) and the inverse-electron-demand Diels-Alder reaction (IEDDA) are the most widely used ones. To accelerate the cycloaddition reaction rates in biological applications, metal catalyst is utilized in CuAAC and strained substrates are used in the SPAAC and IEDDA reactions. However, there are several drawbacks associated with these reactions, for example, the cytotoxicity of metal catalysts, the accessibility and stability of strained alkynes/alkenes. Such issues remain to be addressed.

The in-situ generation of highly reactive species such as nitrile imines, quinone methides or substrates in excited state via photo trigger represents another strategy to develop bioorthogonal cycloaddition reactions. The limited penetration of UV or visible light to deep tissue, as well as the competition reaction on the highly reactive intermediate in complex biological systems posed some restrictions on future applications of these photo trigger-driven bioorthogonal cycloadditions. Continued efforts have been made to develop revised version of known bioorthogonal reactions for specific requirements such as spatial temporal resolution. However, new type of bioorthogonal reaction with mutual orthogonality to the widely used biorthogonal reactions including SPAAC and IEDDA has rarely been reported.

Despite the fact that abundant water exists in biological systems, the exploration of water as a favorable media to promote bioorthogonal cycloadditions has rarely been reported. Based on the nπ* to ππ* state shift of the excited phenanthrenequinone in water that is a polar solvent, the application has recently established a visible light-driven bioorthogonal o-dione and vinyl ether photo-cycloaddition (DVPC) reaction. The application found that the choice of electron-rich C═C bond was the key to the fast electron transfer process from vinyl ether to the excited o-dione at ππ* state. And, the application combined theoretical calculations with rational design to propose the use of the high polarity of water (dielectric constant 78.5) in stabilizing anionic species. The application finds that furan-2(3H)-ones in H₂O can work as highly electron-rich anionic species to undergo a rapid bioorthogonal reaction with the ground-state o-diones. The discovery of this unique driving force represents a new strategy to develop bioorthogonal cycloadditions that could be orthogonal to the widely employed SPAAC and IEDDA reactions. Herein the application reports the metal- and strain-free bioorthogonal o-dione and furan-2(3H)-one cycloaddition (DFC) reaction that can be used together with SPAAC and IEDDA reactions without cross-talking for simultaneous labelling of multiple proteins or live cells.

### The Bioorthogonal Cycloaddition Reaction and Use in Assembly of TAC-Type Molecules

In one aspect, the present application provides a method for preparing (targeting chimera) TAC-type molecule, said method may comprise providing dione-based cycloaddition reaction.

For example, wherein said dione-based cycloaddition reaction may comprise method of the present application.

For example, said TAC-type molecule may comprise proteolysis targeting chimera (PROTAC), autophage targeting chimera (AUTAC), lysosome-targeting chimera (LYTAC), bispecific aptamer chimera, antibody-based PROTAC (AbTAC), covalent nanobody-based PROTAC (GlueTAC) and/or ribonuclease targeting chimera (RIBOTAC).

For example, wherein ligand A with bind affinity to specific target of interest for degradation is linked with first structure comprising a dione group or derivative thereof, and ligand B with function to utilize the inherent degradation pathways for proteins and/or nucleic acids is linked with second structure capable of providing anionic furan-2-olate or derivative thereof.

For example, wherein ligand A with bind affinity to specific target of interest for degradation is linked with second structure capable of providing anionic furan-2-olate or derivative thereof, and ligand B with function to utilize the inherent degradation pathways for proteins and/or nucleic acids is linked with first structure comprising a dione group or derivative thereof.

For example, said method is performed in vivo, in vitro and/or in situ in living cells or on the surface of cells.

For example, said ligand A and said ligand B is linked before or after that said ligand A and/or said ligand B is introduced into living cell. For example, said ligand A and said ligand B is linked before that said ligand A and/or said ligand B is introduced into living cell. For example, said ligand A and said ligand B is linked after that said ligand A and/or said ligand B is introduced into living cell.

For example, said ligand A and said ligand B are introduced into living cell simultaneously or successively. For example, said ligand A and said ligand B are introduced into living cell simultaneously. For example, said ligand A is introduced into living cell firstly and then ligand B is introduced into living cell. For example, said ligand B is introduced into living cell firstly and then ligand A is introduced into living cell.

### The Bioorthogonal Cycloaddition Reaction and Use in Preparation of Bioconjugates

In one aspect, the present application provides a method for preparing bi-functional bioconjugate and/or cyclic peptide, said method may comprise providing dione-based cycloaddition reaction.

For example, wherein said dione-based cycloaddition reaction may comprise method of the present application.

For example, one part of said bi-functional bioconjugate may comprise antibody, peptide other than antibody, nucleic acid, oligosaccharide, lipid, and/or small molecule.

For example, said bi-functional bioconjugate may comprise antibody-peptide conjugate, antibody-nucleic acid conjugate, peptide-nucleic acid peptide, antibody-oligosaccharide conjugate, peptide-lipid conjugate, antibody-small molecule conjugate, and/or nucleic acid-small molecule conjugate.

For example, said cyclic peptide is prepared by linking linear peptide with dione-based cycloaddition reaction.

For example, said cyclic peptide further may comprise first structure comprising a dione group or derivative thereof, and second structure capable of providing anionic furan-2-olate or derivative thereof.

The present application provides a bi-functional bioconjugate and/or cyclic peptide prepared using method of the present application.

The present application provides a method for identification a target, comprising adding a tag on a natural product comprising a dione group or derivative thereof, wherein the tag comprises an anionic furan-2-olate or derivative thereof. The natural product may be a biological sample, optionally, the biological sample comprises protein, peptide or nucleic acid. The natural product may be a biological sample derived from human. The natural product may be a biological sample derived from non-human subject.

The present disclosure further provided the below embodiments:

A method of performing a coupling reaction, comprising providing a first structure comprising a dione group or derivative thereof and a second structure capable of providing anionic furan-2-olate or derivative thereof.

The method of embodiment 1, said second structure comprises a furan-2(3H)-one or derivative thereof.

The method of embodiment 2, said furan-2(3H)-one is optionally substituted; when said furan-2(3H)-one is substituted, said substitution is selected form optional substituents.

The method of any one of embodiments 1-3, said second structure further comprises small molecule, peptide, nucleic acids, peptide nucleic acid (PNA), oligosaccharides, glycans, and/or lipid.

The method of any one of embodiments 1-4, said second structure further comprises therapeutic drug, toxin, and/or detectable substance.

The method of any one of embodiments 1-5, said second structure further comprises antibody, fluorescent protein, membrane bound protein and/or glycoprotein.

The method of any one of embodiments 1-6, said first structure comprising an o-dione group or derivative thereof.

The method of any one of embodiments 1-7, said first structure comprising a phenanthrene-9,10-dione or derivative thereof.

The method of any one of embodiments 1-8, said dione group is optionally substituted; when said dione group is substituted, said substitution is selected form optional substituents.

The method of any one of embodiments 1-9, said first structure further comprises small molecule, peptide, nucleic acids, peptide nucleic acid (PNA), oligosaccharides, glycans, and/or lipid.

The method of any one of embodiments 1-10, said first structure further comprises therapeutic drug, toxin, and/or detectable substance.

The method of any one of embodiments 1-11, said first structure further comprises antibody, fluorescent protein, membrane bound protein and/or glycoprotein.

The method of any one of embodiments 1-12, said method further comprises adding said dione group to said first structure and/or adding said group capable of providing anionic furan-2-olate or derivative thereof to said second structure.

The method of any one of embodiments 1-13, said method is performed at 4° C. to room temperature and/or at pH 5 to 9.

The method of any one of embodiments 1-14, said method is performed for 30 minutes or less.

The method of any one of embodiments 1-15, said method is performed by combining said first structure and said second structure in about 1:100 to about 100:1 ratio.

The method of any one of embodiments 1-16, said method is performed in vitro and/or in vivo.

A combination, comprising a first structure comprising a dione group or derivative thereof and a second structure capable of providing anionic furan-2-olate or derivative thereof.

A method for preparing said combination, comprising linking a first structure with a dione group or derivative thereof and linking a second structure with a group capable of providing anionic furan-2-olate or derivative thereof.

A conjugate, said conjugate is prepared using method of any one of embodiments 1-17.

A kit, comprising combination of embodiment 18, and/or conjugate of embodiment 20.

A method for performing two, three or more bioorthogonal reactions, wherein one of the bioorthogonal reaction comprises dione-based cycloaddition reaction, the dione-based cycloaddition reaction comprises providing a first structure comprising a dione group or derivative thereof and a second structure capable of providing anionic furan-2-olate or derivative thereof.

The method of any one of embodiments 22-23, wherein two, three or more of said bioorthogonal reactions are performed simultaneously in one batch and/or without cross-talking.

The method of any one of embodiments 22-24, wherein two, three or more of said bioorthogonal reactions are performed simultaneously for mutually orthogonal labelling of three types of proteins or three groups of living cells.

The method of any one of embodiments 22-25, said two, three or more bioorthogonal reactions are configured for labelling of two or more proteins and/or live cells.

The method of any one of embodiments 22-26, wherein one of the bioorthogonal reaction comprises cycloaddition.

The method of any one of embodiments 22-27, wherein one of the bioorthogonal reaction comprises Cu-catalyzed azide-alkyne click reaction (CuAAC), the strain-promoted azide-alkyne click reaction (SPAAC) and/or the inverse-electron-demand Diels-Alder reaction (IEDDA).

A cell and/or cell set with structure capable of performing two, three or more bioorthogonal reactions, wherein one of the bioorthogonal reaction comprises dione-based cycloaddition reaction.

A protein and/or protein set with structure capable of performing two, three or more bioorthogonal reactions, wherein one of the bioorthogonal reaction comprises dione-based cycloaddition reaction.

A kit comprising cell and/or cell set of embodiment 29, and/or protein and/or protein set of embodiment 30.

A method for preparing (targeting chimera) TAC-type molecule, said method comprises providing dione-based cycloaddition reaction.

The method of embodiment 32, wherein said dione-based cycloaddition reaction comprises method of any one of embodiments 1-17.

The method of any one of embodiments 32-33, said TAC-type molecule comprises proteolysis targeting chimera (PROTAC), autophage targeting chimera (AUTAC), lysosome-targeting chimera (LYTAC), bispecific aptamer chimera, antibody-based PROTAC (AbTAC), covalent nanobody-based PROTAC (GlueTAC) and/or ribonuclease targeting chimera (RIBOTAC).

The method of any one of embodiments 32-34, wherein ligand A with bind affinity to specific target of interest for degradation is linked with first structure comprising a dione group or derivative thereof, and ligand B with function to utilize the inherent degradation pathways for proteins and/or nucleic acids is linked with second structure capable of providing anionic furan-2-olate or derivative thereof.

The method of any one of embodiments 32-35, wherein ligand A with bind affinity to specific target of interest for degradation is linked with second structure capable of providing anionic furan-2-olate or derivative thereof, and ligand B with function to utilize the inherent degradation pathways for proteins and/or nucleic acids is linked with first structure comprising a dione group or derivative thereof.

The method of any one of embodiments 32-36, said method is performed in vivo, in vitro and/or in situ in living cells.

The method of any one of embodiments 35-37, said ligand A and said ligand B is linked before or after that said ligand A and/or said ligand B is introduced into living cell.

The method of any one of embodiments 35-38, said ligand A and said ligand B are introduced into living cell simultaneously or successively.

A TAC-type molecule prepared using method of any one of embodiments 32-39.

A method for preparing bi-functional bioconjugate and/or cyclic peptide, said method comprises providing dione-based cycloaddition reaction.

The method of embodiment 41, wherein said dione-based cycloaddition reaction comprises method of any one of embodiments 1-17.

The method of any one of embodiments 41-42, one part of said bi-functional bioconjugate comprises antibody, peptide other than antibody, nucleic acid, oligosaccharide, lipid, and/or small molecule.

The method of any one of embodiments 41-43, said bi-functional bioconjugate comprises antibody-peptide conjugate, antibody-nucleic acid conjugate, peptide-nucleic acid peptide, antibody-oligosaccharide conjugate, peptide-lipid conjugate, antibody-small molecule conjugate, and/or nucleic acid-small molecule conjugate.

The method of any one of embodiments 41-44, said cyclic peptide is prepared by linking linear peptide with dione-based cycloaddition reaction. 1

The method of any one of embodiments 41-45, said cyclic peptide further comprises first structure comprising a dione group or derivative thereof, and second structure capable of providing anionic furan-2-olate or derivative thereof.

A bi-functional bioconjugate and/or cyclic peptide prepared using method of any one of embodiments 41-46.

A method for identification a target, comprising adding a tag on a natural product comprising a dione group or derivative thereof, wherein the tag comprises an anionic furan-2-olate or derivative thereof.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like. As for the experimental results (e.g., in a two-sided unpaired Student's t-test), * indicates p<0.05, ** indicates p<0.01, *** indicates p<0.001, *** indicates p<0.0001, and n.s. means not significant.

### Materials and Methods

### General Materials and Methods

All chemicals and solvents were purchased from J&K chemicals (Shanghai, China), Energy Chemical (Shanghai, China), TCI (Shanghai, China) or Sigma-Aldrich (Shanghai, China) unless otherwise indicated. RPMI 1640 Medium, fetal bovine serum (FBS), penicillin/streptomycin, BCA protein assay kit were purchased from Thermo (Shanghai, China). Coomassie staining kit was purchased from Xunbei (Nanjing, China). Bovine serum albumin and lysozyme were purchased from Sangon Biotech (Shanghai, China). Ovalbumin was purchased from Fushen (Shanghai, China). SNAP-GFP was a gift from Prof. Yi Cao' lab at Nanjing University. GDP-Fucose-Az was a gift from Prof. Jie Li' lab at Nanjing University. PE/Cy7-labeled anti-human HER2 antibody and Brilliant Violet 421-labeled anti-human EGFR antibody were purchased from Biolegend (Beijing, China). The cells were ordered from Cell bank of Chinese Academy of Sciences (Shanghai, China).

¹H NMR and ¹³C NMR spectra were obtained on a 400 MHz Bruker AVANCE III-400 spectrometer. Chemical shifts are reported in δ (ppm) relative to the solvent residual peak. Coupling constants are reported in Hz with multiplicities denoted as s (singlet), d (doublet), t (triplet), q (quartet) and m (multiplet). HRMS was done on a Thermo Fisher Q Exactive LC/MS. HPLC was carried out on Agilent 1200 LC with CH₃CN/H₂O (0.1% TFA) as eluents.

### Synthesis and Characterization of FuA and its NHS Ester

### 2,5-dioxopyrrolidin-1-yl 4-(5-oxo-4,5-dihydrofuran-2-yl)butanoate (FuA-NHS)

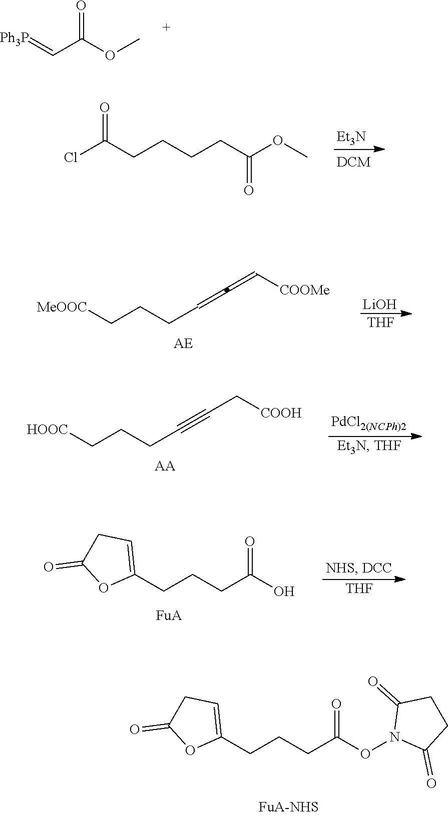

FuA was prepared according to the publication.

### Dimethyl octa-2,3-dienedioate (AE)

Methyl (triphenylphosphoranylidene)acetate (10.0305 g, 30 mmol) was stirred with triethylamine (3.0357 g, 30 mmol) in dry dichloromethane (10 mL per 1 g of acetate) with ice-bath cooling. Methyl 6-chloro-6-oxohexanoate (5.3583 g, 30 mmol) was added dropwise and the mixture was allowed to warm to room temperature then stirred for 12 h. The solvent was removed in vacuo and the residue purified by flash column chromatography (PE:EA=10:1). Colorless oil AE was then obtained (4.8298 g, 81%). ¹H NMR (400 MHz, CDCl₃) δ 5.65-5.57 (m, 2H), 3.74 (s, 3H), 3.67 (s, 3H), 2.40 (dd, J=10.9, 4.4 Hz, 2H), 2.24-2.14 (m, 2H), 1.87-1.75 (m, 2H). ¹³C NMR (101 MHz, CDCl₃) δ 212.43, 173.71, 166.51, 94.52, 88.40, 52.02, 51.58, 33.00, 26.85, 23.76; MS (API) calcd, for C₁₀H₁₅O₄ 199 [M+H]⁺; found 199.

### Oct-3-ynedioic acid (AA)

AE (4.8298 g, 24.37 mmol) was dissolved in tetrahydrofuran (244 mL) and saponified by treatment with aqueous lithium hydroxide (244 mL, 0.5 M) for 30 min. The reaction was monitored by TLC. After completion of the reaction, the mixture was diluted with diethyl ether and water and the layers were separated. The aqueous layer was acidified with 1 M aqueous hydrochloric acid to <pH 1 and extracted with diethyl ether twice (2×10 mL per mmol of material). The combined organic layers were dried over sodium sulfate and concentrated in vacuo to furnish AA (3.3098 g, 80%). ¹H NMR (400 MHz, DMSO) δ 12.28 (s, 2H), 3.24 (t, J=2.4 Hz, 2H), 2.31 (t, J=7.4 Hz, 2H), 2.20 (tt, J=7.0, 2.4 Hz, 2H), 1.64 (p, J=7.2 Hz, 2H). ¹³C NMR (101 MHz, DMSO) δ 174.53, 170.34, 82.40, 74.43, 32.94, 25.95, 24.27, 17.96; MS (API) calcd, for C₈H₉O₄ 169 [M−H]⁻; found 169.

### 4-(5-oxo-4,5-dihydrofuran-2-yl)butanoic acid (FuA)

To a solution of PdCl₂(PhCN)₂ (74.7 mg, 0.1947 mmol) and triethylamine (59.1 mg, 0.5841 mmol) in 40 mL of THF, AA (3.3098 g, 19.47 mmol) was added and the reaction mixture was stirred under 60° C. for 1 h. The whole was concentrated and chromatographed on silica gel (DCM: MeOH=50:1) affording FuA (1.5367 g, 47%). ¹H NMR (400 MHz, CDCl₃) δ 5.21-5.15 (m, 1H), 3.20 (q, J=2.3 Hz, 2H), 2.44 (t, J=7.3 Hz, 2H), 2.42-2.36 (m, 2H), 1.92 (p, J=7.4 Hz, 2H). ¹³C NMR (101 MHz, CDCl₃) δ 178.45, 176.66, 155.88, 99.26, 33.93, 32.78, 27.43, 20.79; HRMS (ESI) calcd, for C₈H₁₀O₄ 169.0506 [M−H]⁻; found 169.0493.

### 2,5-dioxopyrrolidin-1-yl 4-(5-oxo-4,5-dihydrofuran-2-yl) butanoate (FuA-NHS)

FuA (340.3 mg, 2 mmol), DCC (825.3 mg, 4 mmol), NHS (460.4 mg, 4 mmol) were dissolved in 10 mL THF and stirred at room temperature for 4 h. A large amount of white precipitate was generated. The reaction was monitored by TLC. After completion of the reaction, the reaction mixture was filtered to remove the white precipitate and the filtrate was concentrated. Separation by flash column chromatography to obtain FuA-NHS (301.9 mg, 57%). ¹H NMR (400 MHz, CDCl₃) δ 5.25-5.22 (m, 1H), 3.20 (q, J=2.2 Hz, 2H), 2.85 (s, 4H), 2.69 (t, J=7.2 Hz, 2H), 2.46 (ddd, J=7.3, 3.4, 1.3 Hz, 2H), 2.03 (p, J=7.2 Hz, 2H). ¹³C NMR (101 MHz, CDCl₃) δ 176.50, 169.06, 168.01, 155.14, 99.96, 33.95, 29.86, 27.08, 25.60, 20.80; MS (API) calcd. for C₁₂H₁₃NNaO₆ 290 [M+Na]⁺; found 290.

### Preparation and Characterization of the DFC Reaction Products

General preparation procedure: o-dione (0.5 mmol), Furan-2(3H)-one (2.5 mmol) and Et₃N (0.25 mmol) were dissolved in CH₃CN/H₂O (8 mL/2 mL) and the reaction was monitored by TLC. At the end of the reaction, acetonitrile was removed to give white solid precipitated from the system. The white solid was collected and washed with water and ether, dried in an oven to give the product for further characterization.

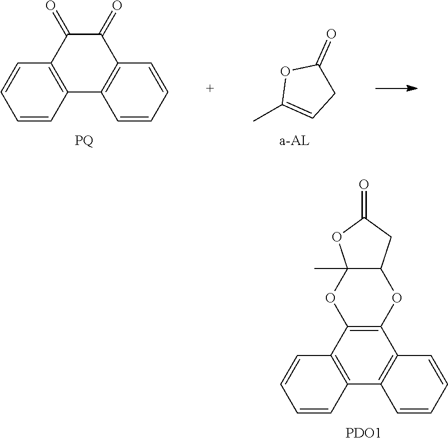

PDO1: white solid, 89%, ¹H NMR (400 MHz, d₆-DMSO) δ 8.82 (d, J=8.4 Hz, 2H), 8.19-8.07 (m, 2H), 7.78-7.60 (m, 4H), 5.27 (dd, J=5.2, 3.3 Hz, 1H), 3.51 (dd, J=17.7, 5.3 Hz, 1H), 3.01 (dd, J=17.7, 3.3 Hz, 1H), 1.93 (s, 3H). ¹³C NMR (100 MHz, d₆-DMSO) δ 171.76, 130.45, 129.44, 127.34, 127.26, 126.67, 126.38, 126.01, 125.78, 124.62, 124.51, 123.10, 120.43, 119.99, 103.30, 73.76, 36.54, 22.47. MS (EI) calcd, for C₁₉H₁₄O₄ 306; found 306.

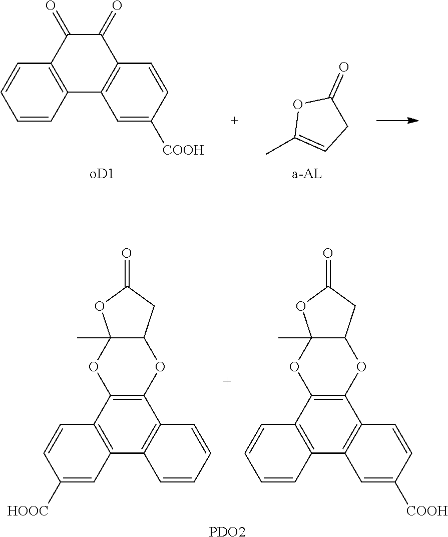

PDO2: light brown solid, 80%, ¹H NMR (400 MHz, d₆-DMSO) δ 13.21 (s, 1H), 9.32 (s, 1H), 8.88-8.81 (m, 1H), 8.25-8.14 (m, 3H), 7.80-7.69 (m, 2H), 5.31 (ddd, J=8.4, 5.2, 3.4 Hz, 1H), 3.53 (ddd, J=17.7, 6.7, 5.3 Hz, 1H), 3.05 (dt, J=17.7, 3.1 Hz, 1H), 1.94 (s, 3H). ¹³C NMR (101 MHz, d₆-DMSO) δ 171.63, 167.38, 132.52, 131.48, 130.15, 129.11, 127.95, 127.90, 127.81, 127.71, 127.38, 127.23, 127.09, 126.80, 126.74, 126.51, 125.98, 125.66, 124.80, 124.69, 124.59, 123.08, 120.89, 120.70, 120.42, 120.27, 103.27, 103.13, 73.96, 73.76, 36.56, 36.35, 22.41; HRMS (ESI) calcd, for C₂₀H₁₄O₆ 349.0718 [M−H]⁻; found 349.0714.

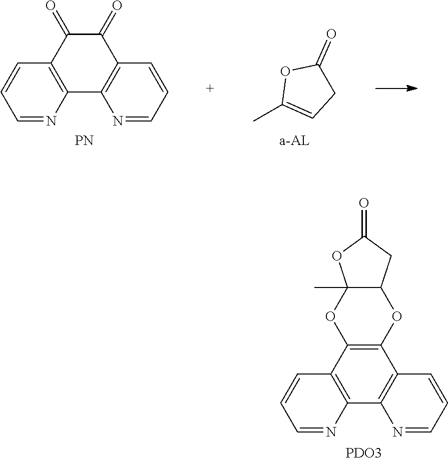

PDO3: white solid, 84%, ¹H NMR (400 MHz, CDCl₃) δ 9.11 (d, J=3.7 Hz, 2H), 8.48 (dd, J=30.7, 8.1 Hz, 2H), 7.64 (dt, J=8.2, 5.9 Hz, 2H), 5.03 (t, J=5.6 Hz, 1H), 3.05 (ddd, J=48.9, 17.5, 5.7 Hz, 2H), 1.93 (s, 3H). ¹³C NMR (101 MHz, CDCl₃) δ 170.31, 149.22, 149.13, 143.06, 142.88, 129.39, 129.12, 128.79, 128.73, 123.12, 123.03, 122.03, 102.06, 74.00, 35.48, 22.62; MS (API) calcd, for C₁₇H₁₃N₂O₄ 309 [M+H]⁺; found 309.

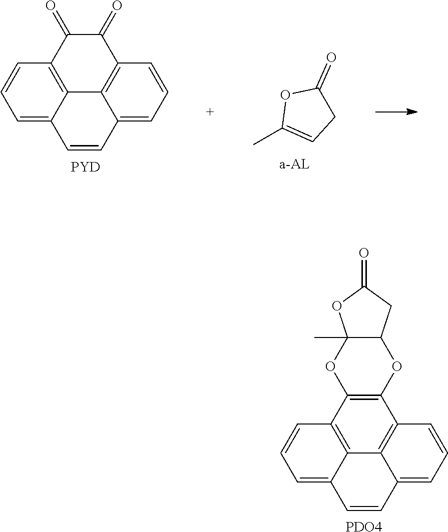

PDO4: white solid, 89%, ¹H NMR (400 MHz, d₆-DMSO) δ 8.39 (d, J=7.8 Hz, 2H), 8.29 (ddd, J=7.7, 3.2, 1.0 Hz, 2H), 8.21 (s, 2H), 8.12 (td, J=7.7, 3.0 Hz, 2H), 5.40 (dd, J=5.3, 3.5 Hz, 1H), 3.57 (dd, J=17.8, 5.3 Hz, 1H), 3.09 (dd, J=17.8, 3.5 Hz, 1H), 2.00 (s, 3H). ¹³C NMR (101 MHz, d₆-DMSO) δ 172.27, 131.86, 130.97, 130.86, 127.91, 127.89, 127.00, 126.91, 125.13, 124.91, 124.36, 124.26, 120.98, 120.72, 118.46, 118.00, 103.84, 74.43, 37.04, 23.03. MS (API) calcd, for C₂₁H₁₄NaO₄ 353 [M+Na]⁺; found 353.

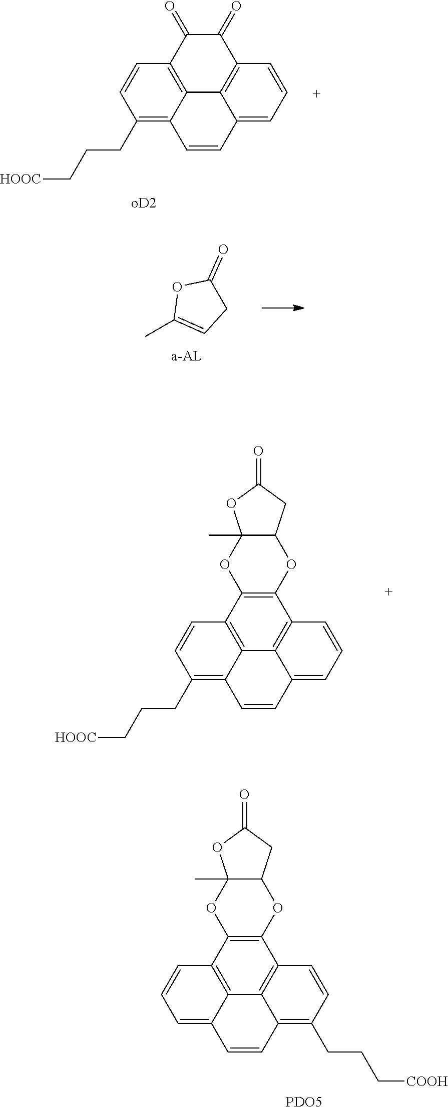

PDO5: light brown solid, 94%, ¹H NMR (400 MHz, d₆-DMSO) δ 12.12 (s, 1H), 8.44-8.30 (m, 3H), 8.30-8.20 (m, 2H), 8.10 (td, J=7.7, 3.0 Hz, 1H), 7.98 (dd, J=8.0, 3.5 Hz, 1H), 5.38 (dd, J=5.2, 3.5 Hz, 1H), 3.56 (ddd, J=17.7, 5.3, 2.4 Hz, 1H), 3.36 (m, 2H), 3.08 (dd, J=17.7, 3.5 Hz, 1H), 2.39 (t, J=7.2 Hz, 2H), 1.99 (m, 5H). ¹³C NMR (101 MHz, d₆-DMSO) δ 174.83, 172.29, 136.39, 136.15, 131.86, 131.11, 130.87, 130.76, 130.15, 128.64, 128.25, 128.17, 127.84, 126.92, 126.84, 124.90, 124.67, 124.50, 124.39, 124.01, 123.01, 122.91, 121.51, 121.33, 121.26, 121.08, 118.31, 117.87, 103.85, 74.44, 74.38, 37.05, 36.97, 33.75, 32.37, 27.34, 23.02; HRMS (ESI) calcd, for C₂₅H₂₀O₆ 415.1187 [M−H]⁻; found 415.1189.

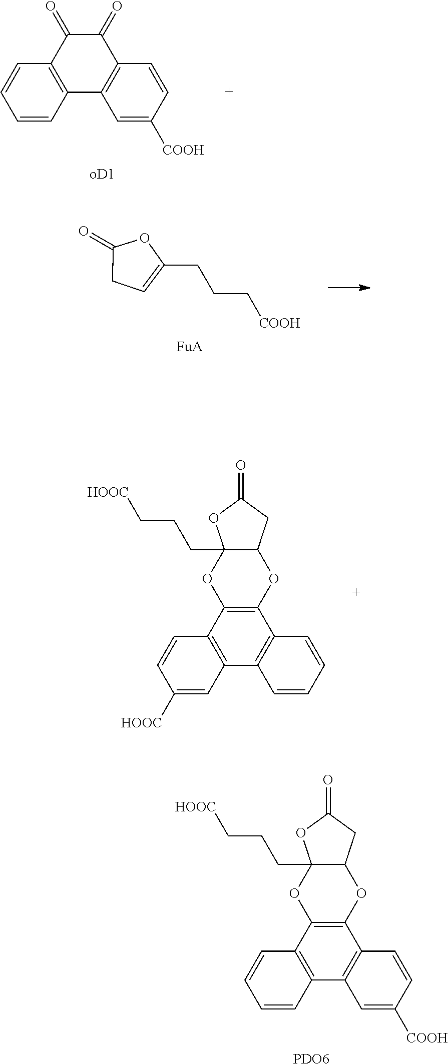

PDO6: white solid, 86%, ¹H NMR (400 MHz, d₆-DMSO) δ 12.67 (s, 2H), 9.32 (s, 1H), 8.85 (d, J=7.6 Hz, 1H), 8.28-8.13 (m, 3H), 7.85-7.65 (m, 2H), 5.46-5.34 (m, 1H), 3.42 (dd, J=11.5, 6.3 Hz, 1H), 3.04 (ddd, J=17.7, 4.6, 2.3 Hz, 1H), 2.37 (t, J=7.3 Hz, 2H), 2.25-2.12 (m, 2H), 2.02-1.79 (m, 2H). ¹³C NMR (101 MHz, d₆-DMSO) δ 174.53, 172.04, 167.88, 132.72, 132.35, 130.36, 130.00, 128.47, 128.31, 128.28, 127.98, 127.81, 127.79, 127.60, 127.32, 127.21, 127.09, 127.03, 126.49, 126.24, 125.40, 125.25, 125.11, 125.07, 123.60, 123.57, 121.38, 121.19, 120.87, 120.71, 104.49, 104.34, 73.58, 73.40, 35.93, 35.74, 34.65, 33.52, 18.19; HRMS (ESI) calcd. for C₂₃H₁₈O₈ 421.0929 [M−H]⁻; found 421.0929.

### Kinetics Studies

All kinetics experiments were carried out at room temperature in 1×PBS (pH=7.4). Second order kinetics were performed by combining o-dione and furan-2(3H)-one in a 1:10 ratio, and the concentration of o-dione is 25/250 μM. Pseudo-first order rate constant k_{obs} was determined by plotting ln[1/(1−x)] versus time and analysis by linear regression. x represents the percent conversion of o-dione. The slope of the linear equation is k_{obs}. The second rate constant k₂ was calculated by k_{obs}/[furan-2(3H)-one]. The reported errors for rate constants are based on the standard deviation of the mean for experiments performed in triplicate.

### Protein Labeling

### Preparation of the Modified Protein BSA-FuA

A solution of BSA (2 mg/mL) in PBS (pH=7.4) was mixed with 200 equivalents of FuA NHS ester and was shaken (900 rpm) at room temperature for 4 h. Unconjugated FuA was removed by repeated rounds of centrifugation using a 10 kDa filter to give purified BSA-FuA solution. The concentration of BSA-FuA was quantified with BCA protein assay kit (Pierce).

### Single-Site Modification of SNAP-GFP

A solution of SNAP-GFP (2 mg/mL) in PBS (pH=7.4) was mixed with 1.5 equivalents of BG-PEG4-oD1 and was incubated at room temperature for 20 min to give oD1 (C156)-SNAP-GFP. For LC-MS/MS detection, 1.5 equivalents of FuA-TAMRA were added into the above solution and incubated for 20 min at room temperature. The obtained protein solutions were subjected directly to LC-MS/MS analysis without further purification. For time-dependent labeling, 1.5 equivalents of FuA-TAMRA were added into the solution of oD1 (C156)-SNAP-GFP and incubated for 0-5 min at room temperature and promptly quenched by 100 equivalents of oD1-PEG₄. The obtained protein solutions were subjected directly to native-PAGE analysis without further purification.

### Preparation of the Modified Protein BSA-TCO, OVA-oD1 and LYSO-Az

A solution of BSA (5 mg/mL) in PBS (pH=7.4) was mixed with 50 equivalents of TCO-NHS and was shaken (900 rpm) at room temperature for 2 h. Unconjugated TCO was removed by repeated rounds of centrifugation using a 30 kDa filter to give purified BSA-TCO solution. For OVA modification, a solution of OVA (5 mg/mL) in PBS (pH=7.4) was mixed with 50 equivalents of oD1-PEG₄-NHS and was shaken (900 rpm) at room temperature for 2 h. Unconjugated oD1 was removed by repeated rounds of centrifugation using a 10 kDa filter to give purified OVA-oD1 solution. As for lysozyme-Az, a solution of lysozyme (10 mg/mL) in PBS (pH=7.4) was mixed with 7 equivalents of Az-PEG₃-NHS and was shaken (900 rpm) at room temperature for 2 h. Then the protein was purified using a 10 kDa ultracentrifuge filter. The concentrations of BSA-TCO, OVA-oD1 and lysozyme-Az were quantified with BCA protein assay kit (Pierce).

### In-Gel Fluorescence Assay

Concentration and Time-Dependent Reaction Between BSA-FuA and oD1-TAMRA

For concentration-dependent reaction, BSA-FuA (2 mg/mL) was incubated with various concentrations of oD1-TAMRA (0-1000 nM) in PBS at room temperature for 30 min. For time-dependent reaction, BSA-FuA (2 mg/mL) was incubated with 100 nM oD1-TAMRA in PBS at room temperature for 0-35 s. Protein samples (20 μg) were further analyzed by gel electrophoresis using 12% polyacrylamide gels and imaged with Bio-Rad Chemdoc imaging system using corresponding filters. Protein loading was assessed by staining with Coomassie Blue according to manufacturer's instructions.

### Single-Site Modification of SNAP-GFP

Protein samples including SNAP-GFP, oD1 (C156)-SNAP-GFP and the reaction product of oD1 (C156)-SNAP-GFP and FuA-TAMRA were analyzed by native-PAGE using 10% polyacrylamide gels and imaged with Bio-Rad Chemdoc imaging system using corresponding filters. Protein loading (20 μg of each sample) was assessed by staining with Coomassie Blue according to manufacturer's instructions.

### Orthogonal Labeling of Different Proteins

For orthogonal labeling of two or three different proteins, the concentrations of BSA-TCO, OVA-oD1 and LYSO-Az were about 0.5 mg/mL, 0.75 mg/mL and 2.5 mg/mL respectively. The concentrations of stock solutions of Tz-Cy5, FuA-TAMRA and DBCO-FITC were 10 mM. For each sample, the corresponding 10 μL of protein solutions and 0.2 μL of stock solutions of related compounds were mixed together and incubated at room temperature for 5 min. Protein samples were further analyzed by gel electrophoresis using 15% polyacrylamide gels and imaged with Bio-Rad Chemdoc imaging system using corresponding filters. Protein loading was assessed by staining with Coomassie Blue according to manufacturer's instructions.

### MTT Assay

SKOV3 cells were seeded in 96-well plates with a concentration of 10,000 cells per well. After 12 h, FuA or PDO6 was added with final concentrations of 0, 40, 80, 120, 160, 200 μM. Then, SKOV3 cells were further cultured for 24 h. MTT assay was then carried out. For each well, 20 μL MTT solution (final concentration, 1 mg/mL) was added and 150 μL DMSO was added after 4 h′ incubation. The absorbance at 490 nm was then measured to indicate the cell viability.

### Live Cell Imaging

### Random Cell Membrane Protein Labeling Using DFC Reaction

SKOV3 cells were seeded in 35 mm glass bottom tissue culture dishes. When reaching 90% confluency, SKOV3 cells were incubated with FuA-PEG₄-NHS (100 μM) for 30 min in PBS buffer and then washed with PBS for three times. The cells with FuA were further incubated with PBS solution of oD1-TAMRA (20 μM) for 10 min and then washed with PBS for three times. After that, the cells were incubated with Hochest 33342 at room temperature for 10 min to stain the nucleus and were then imaged immediately under a Leica confocal microscope with corresponding filters.

### Cell Membrane Glycan Labeling Using DFC Reaction

CHO cells were seeded in 35 mm glass bottom tissue culture dishes. When reaching 90% confluency, CHO cells were incubated with HBSS buffer containing 20 mM MgSO₄, 3 mM HEPES, 0.5% FBS, 100 μM GDP-Fucose-oD1 and 50 μg/mL FucT. After the incubation at 4° C. for 20 min, the cells were washed with PBS (1% FBS contained) three times. The cells with oD1 were further incubated with PBS (1% FBS contained) solution of FuA-TAMRA (20 μM) for 10 min and then washed with PBS (1% FBS contained) for three times. After that, the cells were incubated with Hochest 33342 at room temperature for 10 min to stain the nucleus and were then imaged immediately under a Leica confocal microscope with corresponding filters.

### Negative Controls for Triple Bioorthogonal Cell Labeling

A549, SKOV3 and N87 cells were seeded in 35 mm glass bottom tissue culture dishes respectively. When reaching 90% confluency, A549 cells were incubated with TCO-NHS (50 UM in PBS), SKOV3 cells were incubated with oD1-PEG₄-NHS (50 μM in PBS) and N87 cells were incubated with DBCO-NHS (50 μM in PBS) at room temperature for 20 min. Then the cells were washed with PBS for three times. The obtained A549-TCO cells were further incubated with oD1-TAMRA (20 μM in PBS) or Az-FITC (20 μM in PBS), SKOV3-oD1 cells were incubated with Tz-Cy5 (2 μM in PBS) or Az-FITC (20 μM in PBS), N87-DBCO cells were incubated with Tz-Cy5 (2 μM in PBS) or oD1-TAMRA (20 μM in PBS) at room temperature for 5 min. Then the cells were washed with PBS for three times. After that, all the cells were incubated with Hochest 33342 at room temperature for 10 min to stain the nucleus and were then imaged immediately under a Leica confocal microscope with corresponding filters.

### Orthogonal Labeling of Different Cells and Flow Cytometry

A549, SKOV3 and N87 cells were harvested and washed with PBS twice. Before functional group labeling, A549 cells was stained with Brilliant Violet 421-labeled anti-human EGFR antibody, SKOV3 cells was stained with PE/Cy7-labeled anti-human HER2 antibody and Brilliant Violet 421-labeled anti-human EGFR antibody, and N87 cells was stained with PE/Cy7-labeled anti-human HER2 antibody at 4° C. for 30 min. Then the cells were washed with PBS for three times.

For the treatment group, A549, SKOV3 and N87 cells were incubated with 50 M TCO-NHS, 50 μM oD1-NHS and 50 μM DBCO-NHS respectively in PBS for 20 min in PBS at room temperature to give A549-TCO, SKOV3-oD1 and N87-DBCO. After washing with PBS (1% FBS contained) for three times, the three kinds of labeled cells were mixed and added by Tz-Cy5 (10 mM stock solution in DMSO), FuA-TAMRA (10 mM stock solution in DMSO) and Az-FITC (10 mM stock solution in DMSO) at the final concentration of 2 μM, 20 μM and 20 μM respectively. The mixture was stored at room temperature for 5~10 min and washed twice with PBS (1% FBS contained). For the control group, pre-stained A549, SKOV3 and N87 cells were mixed and added by Tz-Cy5 (10 mM stock solution in DMSO), FuA-TAMRA (10 mM stock solution in DMSO) and Az-FITC (10 mM stock solution in DMSO) at the final concentration of 2 μM, 20 μM and 20 μM respectively. The mixture was stored at room temperature for 5~10 min and washed twice with PBS (1% FBS contained). After all these procedures above, the cells were subjected to flow cytometry analysis with a ACEA NovoCyte Quanteon.

### LC-MS/MS

For in-gel digestion, the protein samples were resolved by SDS-PAGE firstly, and the gel was stained by Coomassie blue. The band of protein was excised, cut into small particles and transferred into pre-cleaned microcentrifuge tube. The resulting gel particles were destained twice with 50% ACN in 25 mM ammonium bicarbonate (ABC), and then dehydrated in ACN. The gel particles were rehydrated with 20 mM DTT in 25 mM ABC and incubated for 45 min at 55° C. Gel particles were washed with 25 mM ABC and dehydrated again with ACN, followed by incubation with 55 mM iodoacetamide in 25 mM ABC for 30 min at room temperature in the dark. The treated gel particles were washed with 25 mM ABC and dehydrated in ACN again. Then the gel particles were rehydrated in a trypsin solution (20 ng/μL) and incubated at 37° C. for 16 h. To extract the tryptic peptides, the gel particles were soaked in ACN/water/FA solution (v:v:v=50:45:5) with a vortex for 30 min. The solution was carefully removed and the extraction was repeated once. The extracts were combined and dried in a vacuum centrifuge.

LC-MS/MS was performed on an Orbitrap Fusion Lumosmass spectrometer (Thermo Fisher Scientific) coupled with an Easy-nLC 1200 LC system. The peptide samples were loaded onto an analytical column (1.9 μm, 120 Å, C18, 250 mm*75 μm i.d.) and eluted with 65 min gradient. The mass spectrometer was performed in data-dependent mode. Full scan spectra were acquired over the m/z range from 350 to 1500 using the Orbitrap mass analyzer. MS/MS fragmentation is performed with HCD mode. The normalized collision energy was 30.

The raw data was analyzed by Pfind3 and searched against bovine proteome in uniprot database or the SNAP-GFP sequence (shown below). Carbamidomethylation of cysteine was set as fix modification. Oxidation of methionine, FuA or FuA+oD1 modification of lysine residues and oD1 or oD1+FuA-TAMRA modification of the C156 residue were set as variable modifications.

The sequence of SNAP-GFP (the bold and underlined marked C represents the modification site C156): (SEQ ID NO: 1)

### Example 1

From the viewpoint of the distortion/interaction model, in the strain-promoted bioorthogonal cycloadditions, the pre-distortion of the 2π cycloaddend dramatically decreases the total distortion energy required for the transition state. In principle, increasing the favorable interaction between the two partners in the cycloaddition transition state is another way to lower the energy barrier. The application noticed that, in our previously reported photo-excited cycloaddition of phenanthrene-9,10-dione with the strain-free alkene, only the electron-rich C═C bond was suitable for achieving fast rate. The applicant found much more electron-rich reaction partners to further enhance the favorable interactions with electron-deficient o-diones in transition states. This strategy may lead to direct use of ground-state o-diones in the cycloaddition, thereby providing a new type of photo trigger-free reaction with strain-free alkenes (FIG. 1). This rational design was further supported by theoretical calculations, as second-order rate constants of the cycloaddition reactions of phenanthrenequinone (PQ) with different alkenes or alkynes were predicted. The application finds that the furan-2(3H)-one, alpha-Angelica lactone (α-AL) with predicted pKa ~12.1 in water due to the aromatization upon deprotonation, whose conjugate base may serve as an anionic cycloaddend for PQ. Owing to the strong electrostatic interactions between the anionic furan-2-olate and the electron-deficient arenes in PQ, the predicted k₂ value for the cycloaddition reaction of PQ and furan-2-olate even reaches up to 7.3×10⁵ M⁻¹ s⁻¹. Taken together, the overall rate constant for the cycloaddition of PQ and α-AL is predicted to be as high as 14.8 M⁻¹ s₋₁ in a buffer solution with pH=7.4 (FIG. 2). According to our calculation, PQ should be inert to α-AL at room temperature without the water media promoted deprotonation process (see SI for details). Moreover, both PQ and α-AL are inert to azides, strained alkynes and alkenes, such as dibenzoazacyclooctyne (DIBAC) and trans-cyclooctene (TCO) according to our computational results. Dimethyl tetrazine is about 1000 times less reactive than PQ to α-AL. Therefore, the o-dione and furan-2(3H)-one cycloaddition (DFC) reaction holds the promise to be a new bioorthogonal reaction that is orthogonal to the strain-promoted cycloadditions such as SPAAC and IEDDA.

Encouraged by the exciting computational results, the application tested the reactions of α-AL with different o-diones (FIG. 2 and FIG. 8A-8B). In phosphate buffered saline (PBS) solution at pH 7.4, phenanthrenequinones (PQ), phenanthrolinequinone PN, and pyrene-diones PYD all showed high reactivities with α-AL. It is noteworthy that Bz, which is not a rigid o-dione, showed no reactivity toward α-AL under the same reaction condition. The reaction of PQ with α-AL does not occur at room temperature in organic solvents, such as benzene and chloroform. The application prepares the carboxylated o-dione oD1 and test its reaction kinetics with α-AL in aqueous solution under different pH ranging from 5 to 9. As shown in FIG. 2, the increase of pH under which α-AL is stable enough apparently accelerates the cycloaddition reaction. All these experimental results may be consistent with computational prediction and indicate that the conjugate base of α-AL, furan-2-olate, is the real reactive species toward o-diones.

The application then synthesized the furan-2(3H)-one substrate (FuA) with a carboxylic acid group and tested the reaction with o-dione oD1 in PBS. The bioorthogonal DFC reaction of oD1 with FuA proceeded rapidly with a second-order constant about 4 M⁻¹ s⁻¹ in pH 7.4 PBS buffer and the reaction gave a pair of regioisomers in high yield (FIG. 3A and FIG. 8A-8B). Next, the application tested the DFC reaction on bovine serum albumin (BSA) protein modified with FuA (BSA-FuA) on the side chain of Lysine with PQ as well as oD1-TAMRA. BSA-FuA incubated with PQ in PBS showed fluorescence turn-on due to the formation of the PDO type cycloadduct, which is confirmed by LC-MS/MS analysis of BSA-FuA before and after incubation with PQ (FIG. 9B). Using oD1-TAMRA with strong fluorescence signal for ultra-sensitive detection in gel electrophoresis assay, the application found that the DFC reaction on proteins occurred rapidly at substrate concentration as low as nM level (FIG. 9D). Time dependence of BSA-FuA with OD1-TAMRA was then examined. Since the reaction proceeded spontaneously and rapidly without any external trigger, the application used excess amount of free FuA to quench the reaction on proteins at different time points before the mixtures were subject to gel electrophoresis assay. The results showed that the DFC reactions on proteins proceeded with ultra-fast kinetics and completed within 1 min (FIG. 3B).

The application also realized single-site modification of protein using the DFC reaction. Engineered GFP protein was fused with SNAP tag (SNAP-GFP) that can react readily with the nucleobase O⁶-benzylguanine (BG) derivatives via the reaction of the C156 of the SNAP tag with BG (FIG. 3C). The application firstly linked oD1 with BG via PEG linker to give BG-PEG₄-oD1, which further reacted with SNAP-GFP to generate oD1 (C156)-SNAP-GFP with the o-dione moiety presented at the specific site of the fusion protein. Upon incubation of oD1 (C156)-SNAP-GFP with FuA-TAMRA (the chemical structure is shown in FIG. 9C), the fusion protein was rapidly labelled with fluorescence and the fluorescence intensity reached saturation within 3 min as indicated in the native-PAGE gel electrophoresis with overlaid red fluorescence signal from TAMRA and green fluorescence signal from GFP (FIG. 10). The application also verified the DFC reaction on the C156 residue of the fusion protein using LC-MS/MS analysis. The mass spectrometry samples were processed by the aforementioned method. As shown in FIG. 3D, for oD1 (C156)-SNAP-GFP, the peptide containing C156 can be detected with oD1 modified fragment peaks (blue peaks). After incubating with FuA-TAMRA, fragment peaks modified with TAMRA through DFC reaction was detected (purple peaks).

Ideal bioorthogonal reactions should be able to proceed smoothly with fast kinetics and high efficiency in living systems without significant interference on physiological activities. To investigate whether the DFC reaction could meet the stringent requirements of bioorthogonal reactions, further studies of the reaction were conducted. The application used two different methods to load FuA or oD1 as the bioorthogonal handle on live cells. Firstly, using the NHS ester of FuA (FuA-NHS) to react with free amine group on the membrane proteins, FuA was attached to the membrane proteins of SKOV3 cells as the bioorthogonal handle subject to the DFC reaction (FIG. 11A). The FuA loaded cells were then incubated with oD1-TAMRA (FIG. 4A). As shown in FIG. 4B, cells with FuA attached were rapidly labelled by oD1-TAMRA, while control cells without pre-modification with FuA gave minimal background fluorescence signal.

Secondly, the o-dione oD1 was attached to the cell membrane glycan of CHO cells using the chemoenzymatic glycan labelling probe GDP-Fucose-oD1. According to the protocol, GDP-Fucose-oD1 was synthesized by copper (I)-catalyzed click reaction between GDP-Fucose-Azide and alkyne modified oD1 (oD1-Al) (FIG. 12). After quenching the reaction with the copper chelator bathocuproine sulfonate (BCS), the crude product was directly transferred by α1,3 FucT onto the cell surface to obtain oD1 attached cells (FIG. 11B). Afterwards oD1 attached cells were incubated with FuA-TAMRA (FIG. 4C) and fluorescently labelled rapidly within 10 min (FIG. 4D).

The application also tested the cytotoxicity of key compounds related to the DFC reaction. The o-dione oD1 was known to be compatible with live cells. The furanone derivative FuA, as well as the cycloadduct of FuA with oD1, also showed good compatibility with live cells according to the methylthiazolyldiphenyl-tetrazolium bromide (MTT) assay (FIG. 13). These results confirmed the rapid and biocompatible labelling of proteins and glycans on live cells by the DFC reaction.

Expanding the bioorthogonal tool-box with mutually orthogonal bioorthogonal cycloadditions is of great importance. However, only a few examples of triple orthogonal labelling of biomolecules may have been reported to date. In light of the unique mechanism of the DFC reaction, it is conceivable that the reaction could be used in combination with strain-promoted bioorthogonal cycloadditions such as the SPAAC or IEDDA reaction to label multiple biomolecules simultaneously without cross-talking. To prove the feasibility of the strategy, the application then tried the DFC reaction together with SPAAC or/and IEDDA for simultaneous labelling of two or three proteins. As shown in FIG. 5, Bovine serum albumin (BSA), ovalbumin (OVA) and lysozyme (LYSO) were firstly modified with trans-cyclooctene (TCO), oD1 or azide groups as bioorthogonal handles respectively to give BSA-TCO, OVA-oD1 and LYSO-Az (FIG. 14). Phenyl-methyl-tetrazine (Tz), FuA and benzoannulated strained alkynes (DBCO) were modified with three fluorescent dyes Cy5, TAMRA and FITC to give Tz-Cy5, FuA-TAMRA and DBCO-FITC respectively. BSA-TCO, OVA-oD1 and LYSO-Az were reacted with different fluorophore-modified bioorthogonal compounds and then analyzed by sodium dodecyl sulfate-polyacrylamide gel electrophoresis (SDS-PAGE). Fluorescent signals of the protein bands were used to reflect the protein modification.

Orthogonal protein labelling using IEDDA, DFC or SPAAC reaction were firstly detected. As shown in FIG. 5 and FIG. 15, after mixing with FuA-TAMRA, the OVA-oD1protein band in lane 2 was detected by TAMRA signal, which indicates successful labelling of OVA-oD1 through DFC reaction. Similarly, lane1 and lane 3 verified successful labelling of BSA-TCO and LYSO-Azutilizing IEDDA or SPAAC reaction. The application then applied the combination of two of the three reactions for orthogonal labelling of two proteins (FIG. 15). The results (lanes 4-6) showed no cross-talk signal of these combinations. Based on the above results, the application further applied DFC, IEDDA and SPAAC reactions for simultaneous labelling of three proteins. The mixture of Tz-Cy5, FuA-TAMRA and DBCO-FITC was added into the premixed protein solution containing BSA-TCO, OVA-oD1 and LYSO-Az and incubated at room temperature for 5 min. The result in lane 7 showed no cross-talk signal, which confirms that the DFC reaction can be used together with the SPAAC and IEDDA reactions to label multiple biomolecules simultaneously without cross-talking.

Encouraged by the successful labelling of three proteins without cross-talking based on the DFC, SPAAC and IEDDA reactions, the application further explored the mutually orthogonal labelling of three different live cells. To construct a model system for orthogonal labelling, the application chose A549, SKOV3 and N87 cells as the bioorthogonal functional group carriers and flow cytometry as the test method. A549, SKOV3 and N87 cells were modified with TCO, oD1 and DBCO respectively to give A549(TCO), SKOV3(oD1) and N87(DBCO) cells. The application anticipated that A549(TCO), SKOV3(oD1) and N87(DBCO) should be fluorescently labelled by Tz-Cy5, FuA-TAMRA and Az-FITC without cross-talking (FIG. 6A). In this case, A549(TCO), SKOV3(oD1) and N87(DBCO) cells should be detected only through the fluorescence of Cy5, TAMRA and FITC, respectively, by flow cytometry.

To verify this hypothesis, the application incubated A549, SKOV3 and N87 cells with TCO NHS ester, oD1 NHS ester and DBCO NHS ester respectively (FIG. 16). The obtained A549(TCO), SKOV3(oD1) and N87(DBCO) cells were mixed together and incubated in PBS buffer containing Tz-Cy5, FuA-TAMRA and Az-FITC for 5 min. As the control group, non-labelled A549, SKOV3 and N87 cells were also mixed and incubated in PBS buffer containing Tz-Cy5, FuA-TAMRA and Az-FITC for 5 min. Then flow cytometry was performed. In addition, for the convenience of analysis, the three cells were pre-labelled by HER2 and EGFR fluorescent antibody to be divided into three groups, which is consistent with the three gates shown in FIG. 6B.

The application analyzed the fluorescence signals of the cells in the gate “A549”. As shown in FIG. 6C, compared with the control group, fluorescent intensity of the treatment group greatly shifted on Cy5 channel but not on TAMRA channel nor FITC channel, which indicates A549(TCO) can only be modified by the IEDDA reaction. Using the same analytical method, the application can conclude that SKOV3 (oD1) can only be modified by the DFC reaction and N87(DBCO) can only be modified by the SPAAC reaction in the mixed system. Statistical results of fluorescence intensity are shown in FIG. 6D. In addition, the application incubated A549(TCO) with FuA-TAMRA or Az-FITC, SKOV3(oD1) with Tz-Cy5 or Az-FITC and N87 (DBCO) with Tz-Cy5 or FuA-TAMRA respectively and imaged the cells using a confocal microscope. The results showed that no obvious labelling signal was detected (FIGS. 17A-17F). These results together suggest great potential of the DFC reaction to develop versatile and robust bioorthogonal tools to be used alone or together with classic bioorthogonal reactions in living systems.

### Conclusion of the Example

In summary, the application developed a metal- and strain-free bioorthogonal DFC reaction. The reaction was designed on the basis of a new interaction-enhanced strategy, and it is distinct from the widely adopted strain-promoted approaches. Furan-2(3H)-ones do not directly participate in the DFC reaction as the 2π cycloaddend, and the real reactive species towards o-diones are anionic furan-2-olates. The aromaticity of furan makes it relatively easy to form this unusual anionic alkene in water, showing very strong electrostatic interaction with the electron-deficient fused arenes in o-diones. The distinct reaction pathway enables the DFC reaction to be used in combination with other bioorthogonal cycloadditions such as SPAAC and IEDDA for mutually orthogonal labelling of multiple biomolecules. The application demonstrated simultaneous labelling of multiple proteins or live cells without cross-talking by using three bioorthogonal SPAAC/DFC/IEDDA reactions. This strategy will provide new avenues for the design and development of new bioorthogonal reactions as powerful and versatile tools in future in vivo applications.

### Example 2

### In Vivo Labeling

1: Cells pre-labeled with F as one of the bioorthogonal functional group in the DFC (o-dione and furan-2(3H)-one cycloaddition) reaction are injected subcutaneously into living animal such as mice. Molecular imaging tracer (including fluorescent dye, MRI imaging tracer, PET or SPECT imaging tracer, F19 tracer, etc.) with chemically modification to install the other bioorthogonal handle o-dione (D-tracer) is then administrated in-demand into living animal. The DFC reaction between the F-labelled cells and the D-tracer in the living animal forms covalent bond between the cells and the tracer and traps the tracer much longer for further imaging purpose to give high imaging signal on the target cells to background signal under imaging instruments such as the optical imaging, MRI imging, PET imaing, SPECT imaging etc.

2: Cells pre-labeled with o-dione (D) as one of the bioorthogonal functional group in the DFC reaction are injected subcutaneously into living animal such as mice. Molecular imaging tracer (including fluorescent dye, MRI imaging tracer, PET or SPECT imaging tracer, F19 tracer, etc.) with chemically modification to install the other bioorthogonal handle F (F-tracer) is then administrated in-demand into living animal. The DFC reaction between the D-labelled cells and the F-tracer in the living animal forms covalent bond between the cells and the tracer and traps the tracer much longer for further imaging purpose to give high imaging signal on the target cells to background signal under imaging instruments such as the optical imaging, MRI imaging, PET imaging, SPECT imaging etc (FIG. 18).

Hela cells were cultured and divided into 3 groups, then cultured with AL-NHS or controls for 30 min. After incubation the cells were digested with trypsin and washed with PBS for three times. Cells were then cultured with PQ-Cy5.5 or controls for 10 min (FIG. 19).

Hela cells labeled with AL were injected subcutaneously into the left side of the mouse, unlabeled Hela cells were injected into the other. PQ-Cy5.5 (70 μM,200 μL) was injected in vein 15 min later, and imaged at different time points (FIG. 20).

### Example 3

### Assembly of Bifunctional Chimeras Based on the DFC Reaction

Bifunctional chimeras contain two functional ligands: ligand A to bind with target of interest for degradation, ligand B to hijack the inherent degradation pathways for proteins or nucleic acids in cells. Only when ligand A and ligand B are linked with each other to form the chimeras, the function to degrade the specific target of interest is activated. Different bifunctional chimeras, including the proteolysis targeting chimeras (PROTACs), the autophage targeting chimeras (AUTACs), the lysosome-targeting chimeras (LYTACs), the bispecific aptamer chimeras, the antibody-based PROTACs (AbTACs), the covalent nanobody-based PROTACs (GlueTACs) and the ribonuclease targeting chimeras (RIBOTACs) can be assembled using the DFC reaction in vitro or in situ in living cells.

General protocol for in vitro assembly of bifunctional chimeras: ligand A with known bind affinity to specific target of interest for degradation is modified with F as one of the bioorthogonal functional group in the DFC reaction to give A-F. Ligand B with known function to hijack the inherent degradation pathways for proteins or nucleic acids is linked with the other bioorthogonal handle o-dione (B-D). Using the DFC reaction, different bifunctional chimeras (A-linker-B) are generated in reaction buffer. A-linker-B when treated with cells is able to induce the degradation of specific target of interest in live cells. The other way, use A-D and B-F for DFC reaction is also able to generate A-linker-B (FIG. 21).

General protocol for in situ assembly of bifunctional chimeras inside cells: A-F is firstly treated with live cells to bind with specific target of interest in live cells. Upon the introduction of B-D into the same cell, intracellular DFC reaction resulted in the formation of A-linker-B for target degradation inside cells. The other combination is A-D and B-F (FIG. 22).

### Example 4

### DFC-Based Bioconjugation to Prepare Drug Conjugates

1: General idea to prepare drug conjugates containing two functional units: the two functional units are linked together with covalent bond by DFC reaction as illustrated below. One functional unit is modified with F, the other with D. The integration of the two functional units is realized by DFC reaction in bio-compatible conditions. Antibody-peptide conjugates, antibody-nucleic acid conjugates, peptide-nucleic acid peptides, antibody-oligosaccharide conjugates, peptide-lipid conjugates, antibody-small molecule conjugates, nucleic acid-small molecule conjugates are included (FIG. 23).

2: General idea to prepare drug conjugate containing cyclic peptides. The D modified peptide was obtained by standard solid-phase synthesis, and the exposed amino group obtained after removal of protection could be modified by F-NHS and then conjugated to obtain cyclic-peptide using DFC reaction (FIG. 24 and FIG. 25).

Peptide substrate

NH₂-Gly-Phe-Tyr-Lys
NH₂-Ala-Gln-His-Dap
NH₂-Arg-Gly-Asp-Lys
NH₂-DArg-Gly-DAsp-Lys
NH₂-Ala-Met-Arg-Gly-Dap
NH₂-Thr-Val-Trp-Ala-Dap
NH₂-Thr-Val-Trp-Ala-Dap-Phe-Ala
NH₂-Glu-Leu-Pro-Ser-Lys

Cyclic-peptide modified with D/F can be conjugated using DFC reaction (FIG. 26).

Cyclic-peptide modified with F and azide can be orthogonal coupled to two different cyclic-peptides modified with D/Dibenzocyclooctyne respectively (FIG. 27).

### Example 5

### DFC-Based Bioconjugation to Prepare TAC-Type Molecule

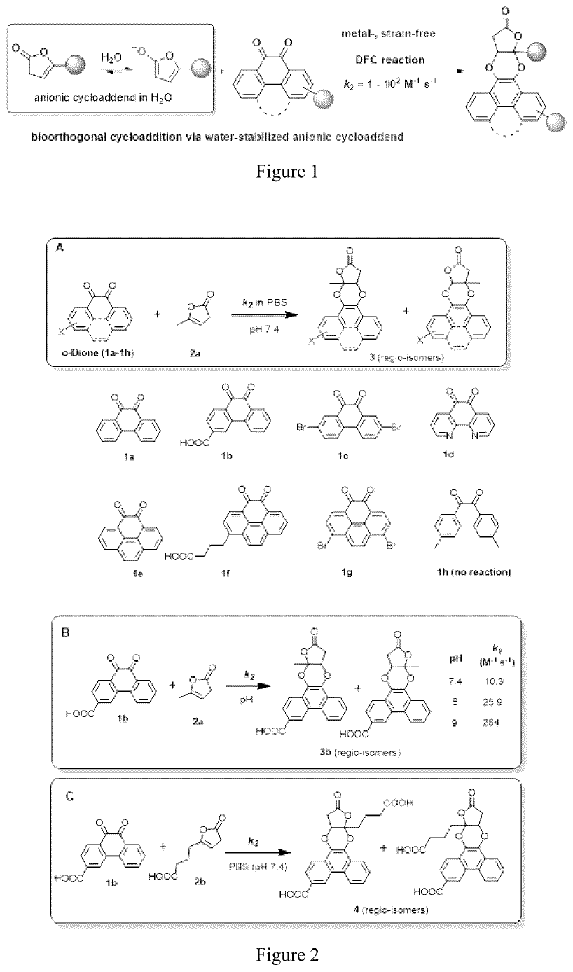

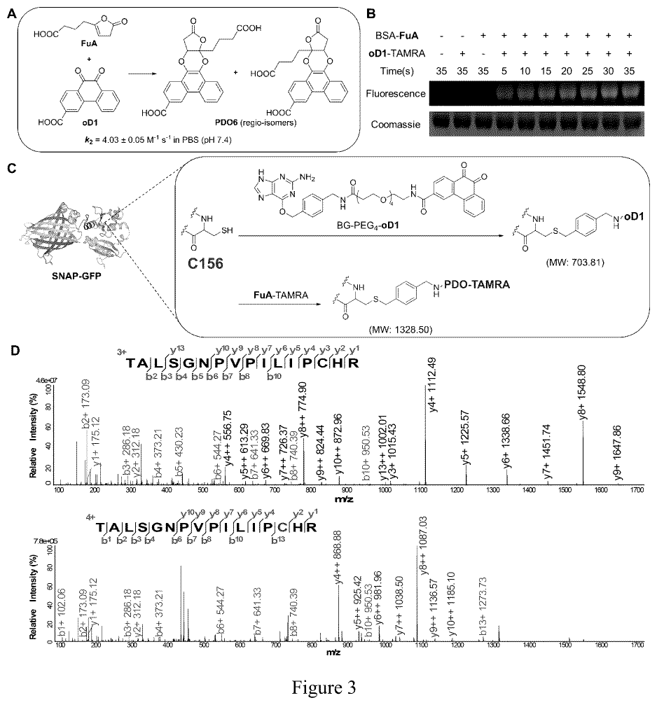

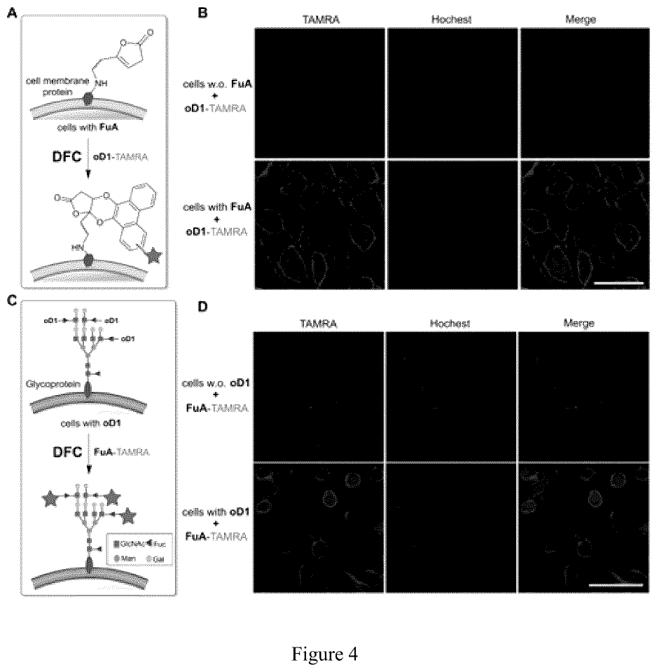

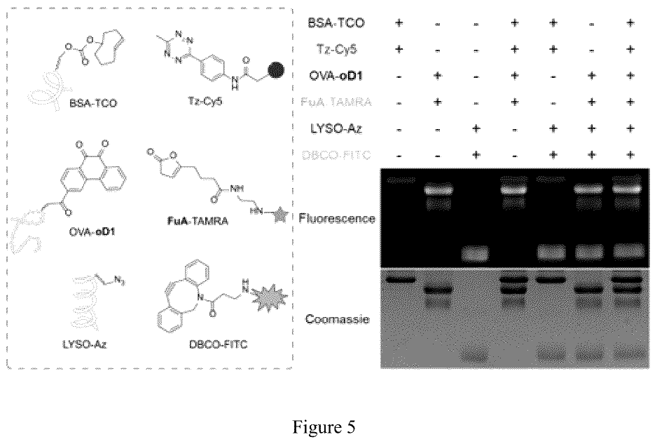

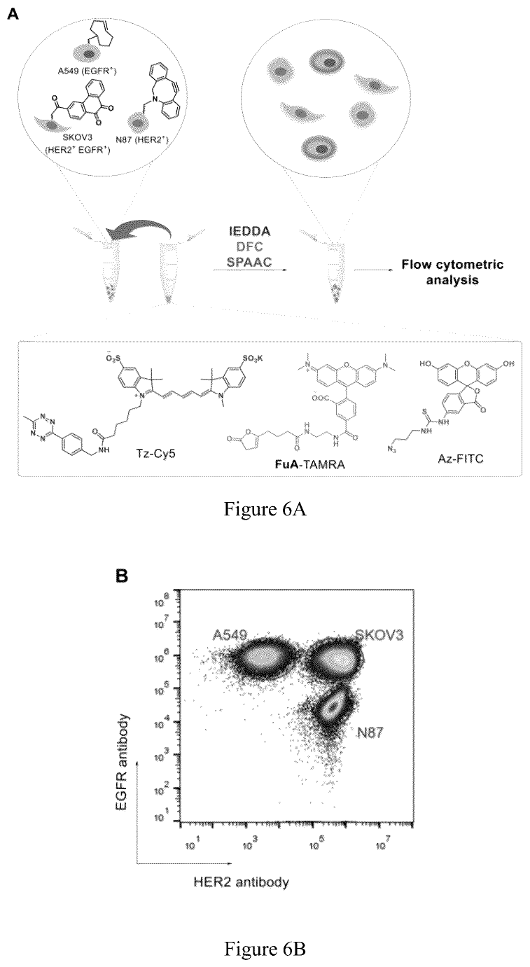

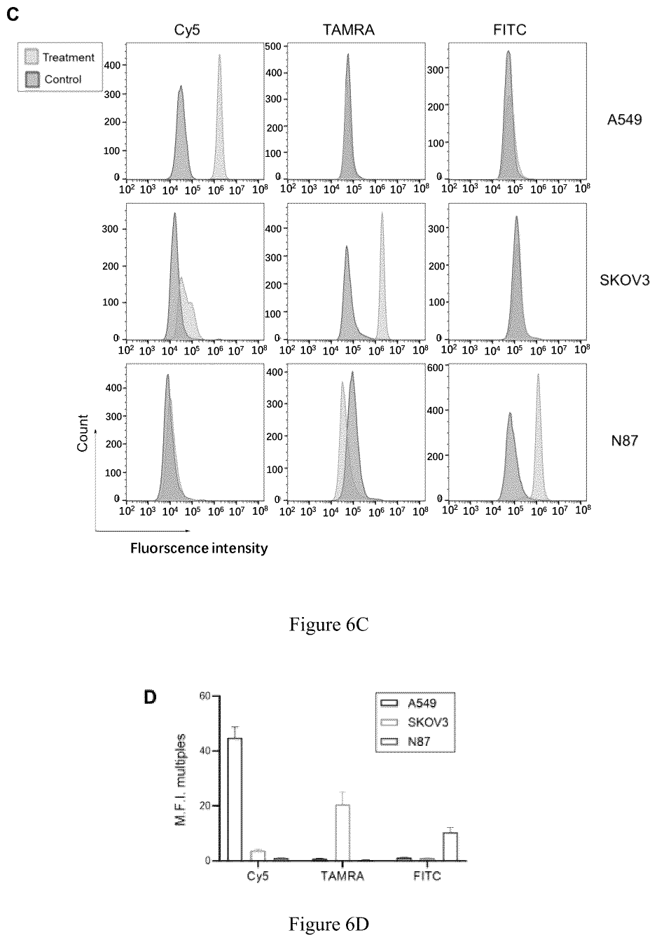

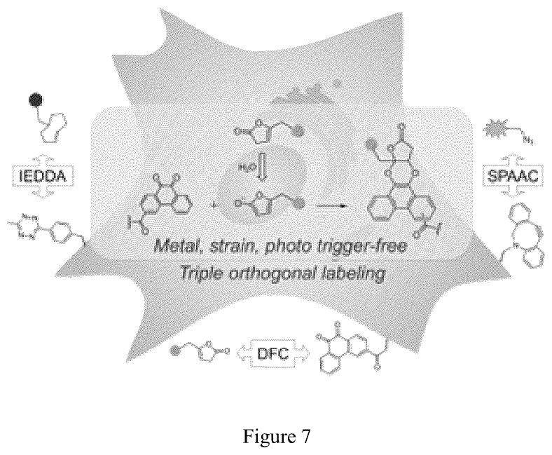

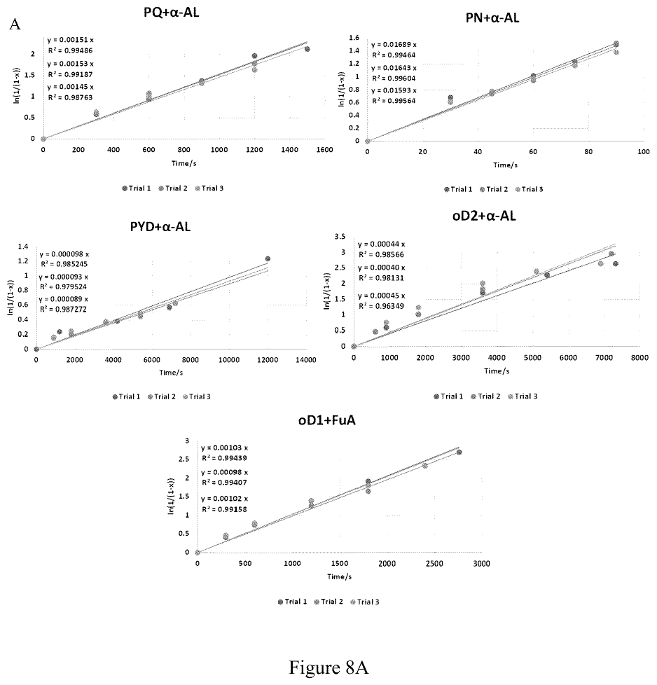

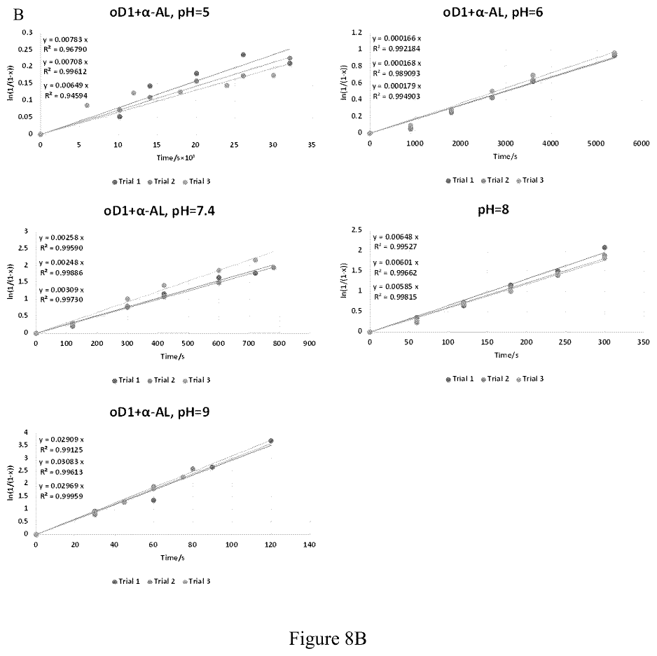

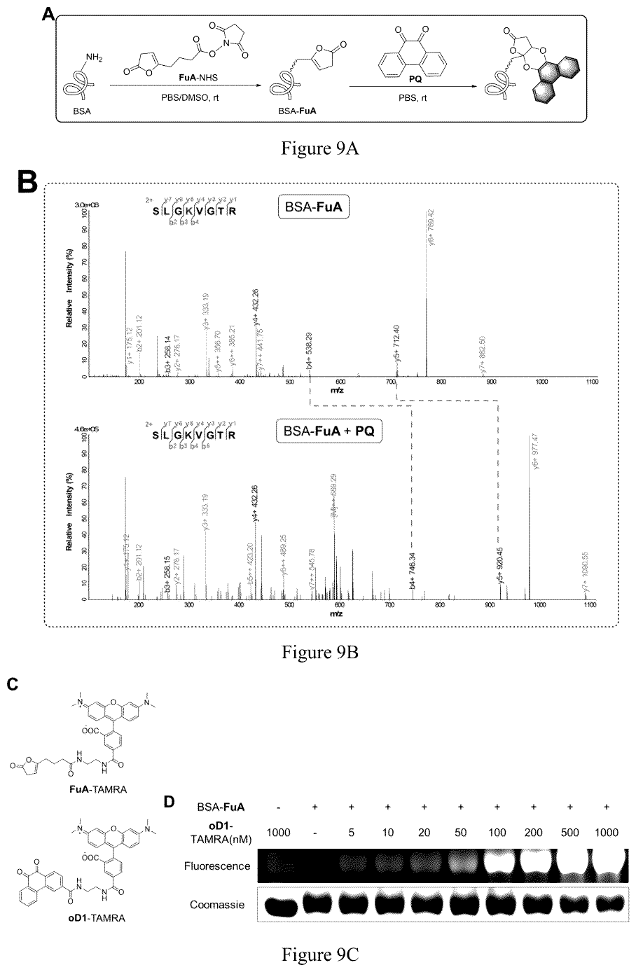

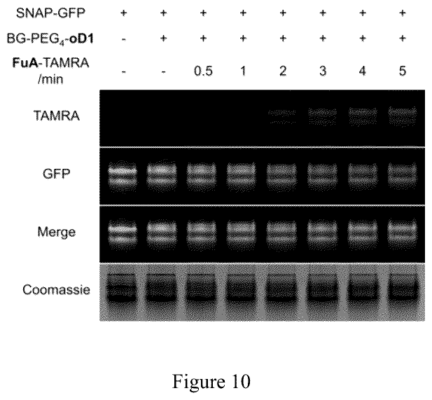

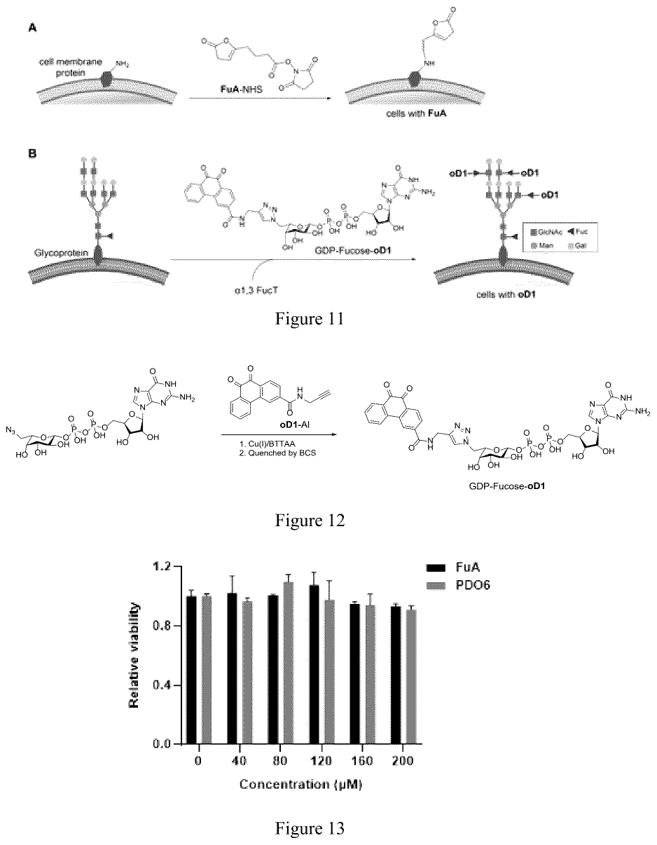

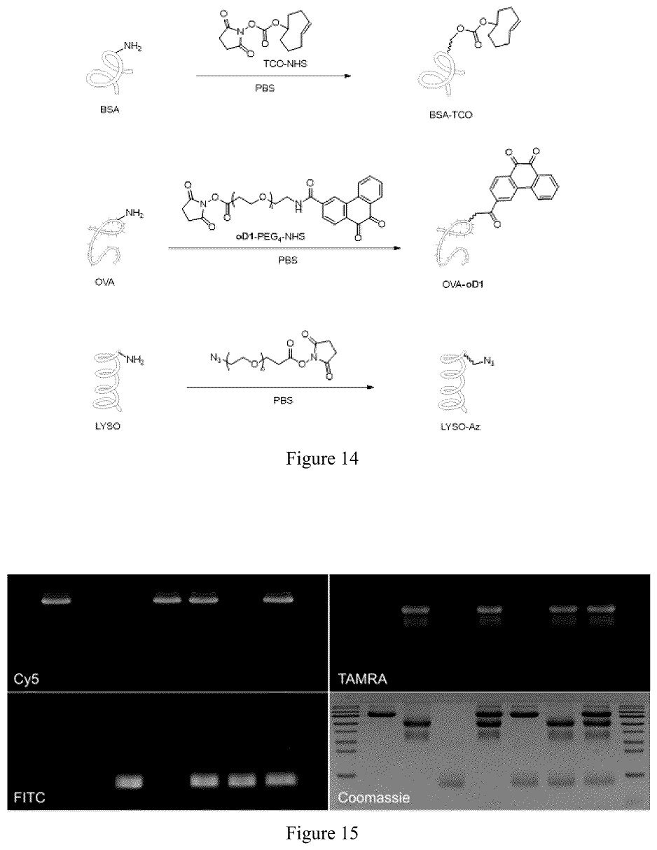

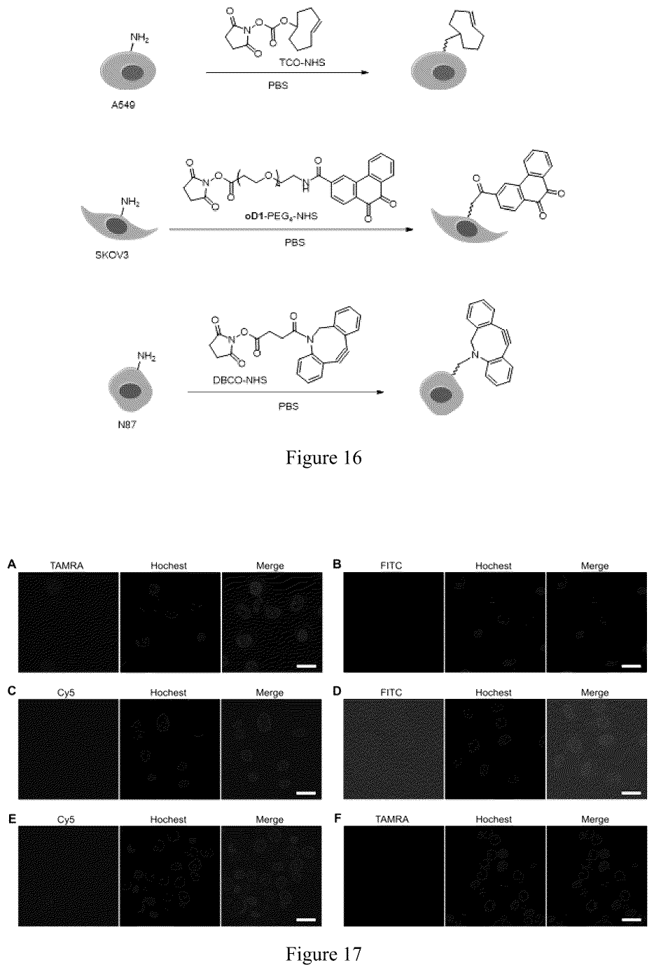

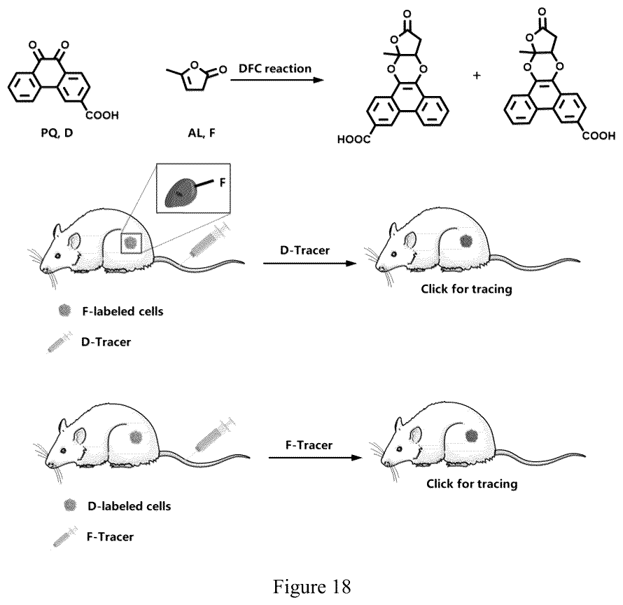

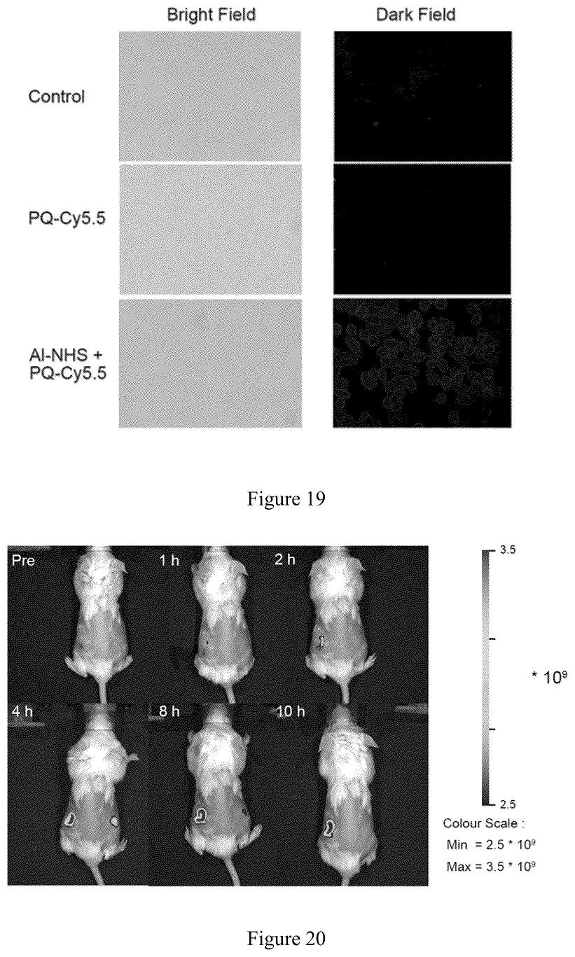

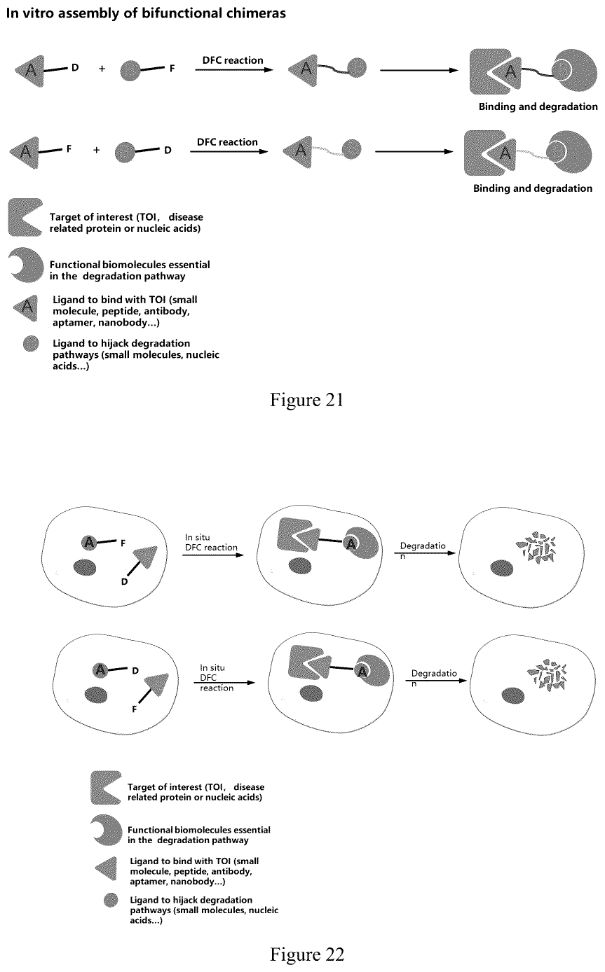

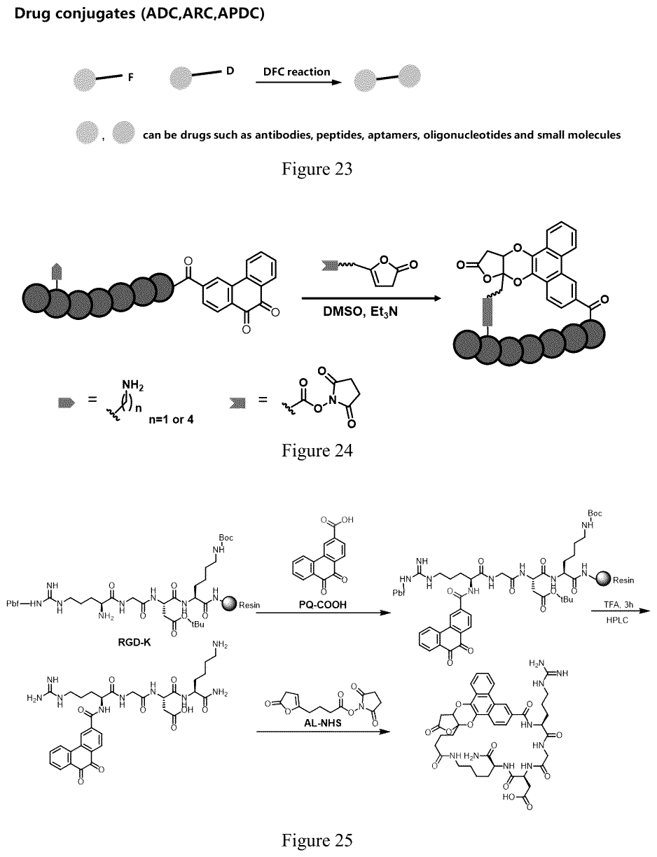

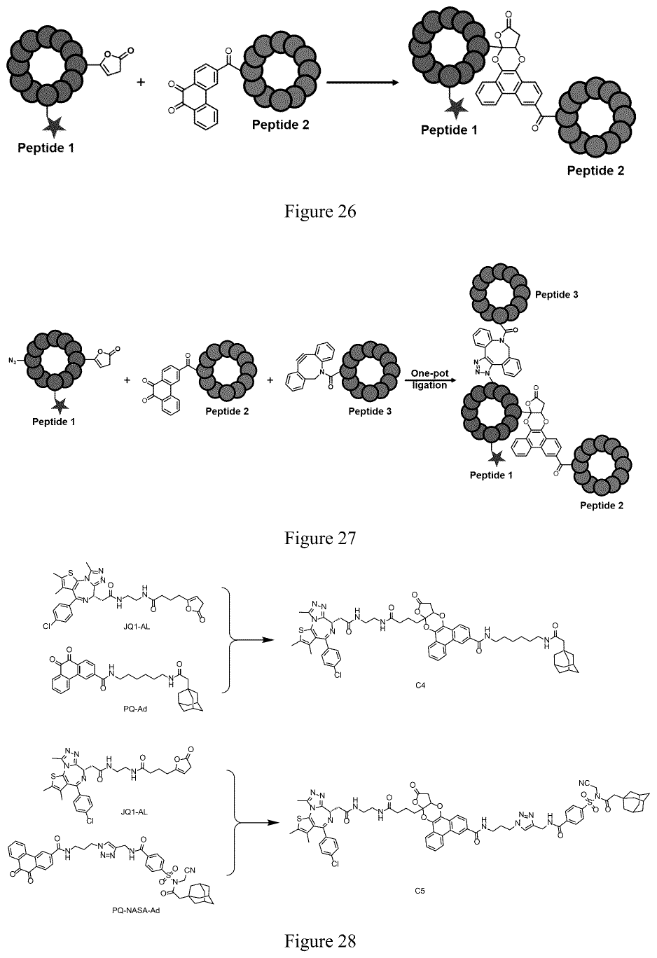

Connecting non-covalent ligand JQ1 of BRD4 to α-angelactone to synthesize DFC-reaction product. JQ1 was linked with 9,10-phenanthrenequinone through a NASA fragment capable of adjacent covalent label or a linker. BRD4 targeting molecules PQ-Ad, PQ-NASA-Ad and JQ1-AL were synthesized through an amide condensation reaction and monovalent copper-catalyzed click reaction coupling. The DFC reaction is shown in FIG. 28.

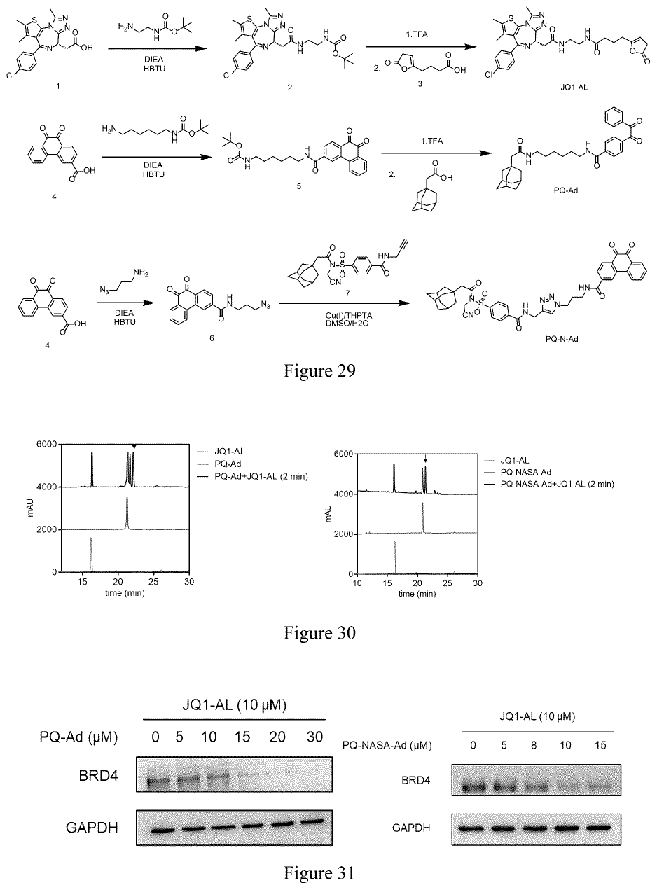

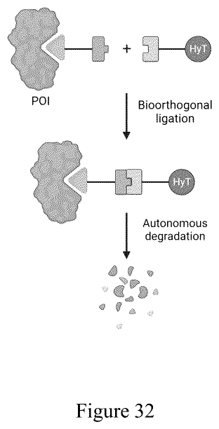

The synthesis steps of PQ-NASA-Ad are shown in FIG. 29: Compound 1 (1 eq) and monoboc ethylenediamine (2 eq) are added to benzotriazole-N,N,N′,N′-tetramethylurea hexafluoro phosphate (HBTU, 2,5 eq) and N, N-diisopropylethylamine (DPEA, 2.5 eq). The mixture was dissolved in DMF and reacted at room temperature for 12 hours to obtain Compound 2. After reacting at room temperature for 4 hours, Compound 3 (2 eq), HBTU (2 eq), and DPEA (2.5 eq) were added, the reaction was carried out at room temperature for 12 hours, and the purified product JQ1-AL was obtained by column chromatography. Compound 4, Compound 5, and Compound 6 undergo amide condensation with PQ-Ad, and the synthesis steps are the same. Molecules 6 and 7 (1 eq), copper sulfate (2 eq), sodium ascorbate (2 eq), tris(3-hydroxypropyltriazolylmethyl)amine (THPTA, 2 eq) were dissolved in H₂O/dimethyl Chemistry sulfone (DMSO). The reaction was carried out at room temperature for 4 hours and then purified by high performance liquid chromatography to obtain the product molecule PQ-NASA-Ad.

### Detection of DFC Reactions in Solution.

After reacting 200 μM compound JQ-AL and 200 μM PQ-Ad or PQ-N-Ad in PBS buffer solution for 2 min, it was analyzed by high performance liquid chromatography. As shown in FIG. 30, the product peaks generated by the two groups of compound molecules are respectively generated after the reaction. The results show that compounds based on DFC reaction react quickly and efficiently in solution.

### Detection Activity of DFC Reaction in Cell

Add 10 μM compound JQ1-AL to MV4-11 cells, incubate for 6 hours, add different concentrations of PQ-NASA-Ad or PQ-Ad, and then incubate for 18 hours for western blot detection. The results are shown in FIG. 30. The results shows that BRD4 in cells was significantly down-regulated as the incubation time increased. Compound 1 of different concentrations were added to MV4-11 cells and incubated for 18 hours. The results of Western blotting are shown in FIG. 31. The intracellular BRD4 degradation effect increased as the concentration of the compound increased.

The result shows that the molecule synthesized through intracellular DFC reaction can target and achieve BRD4 degradation in MV4-11 cells.

### Example 6

### DFC-Based Bioconjugation to Prepare RNase-RNA Binder Molecule

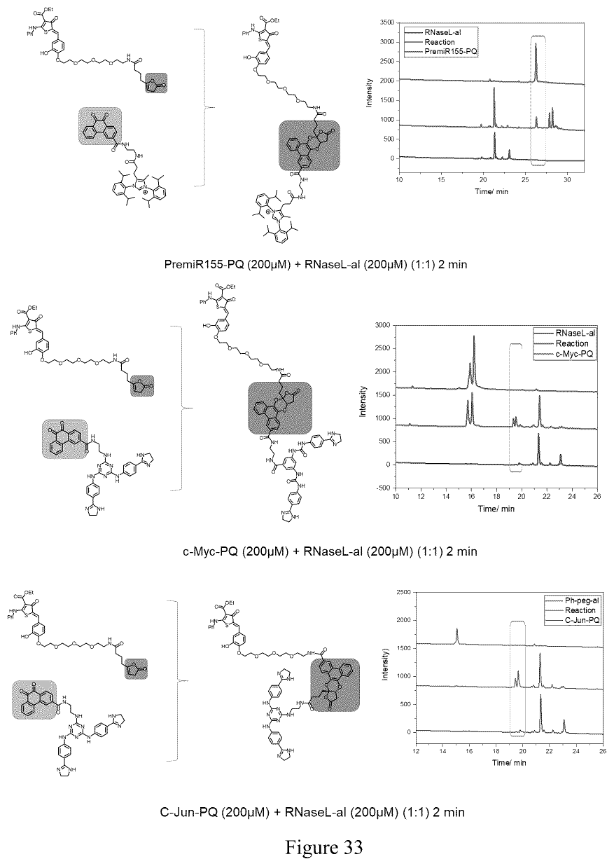

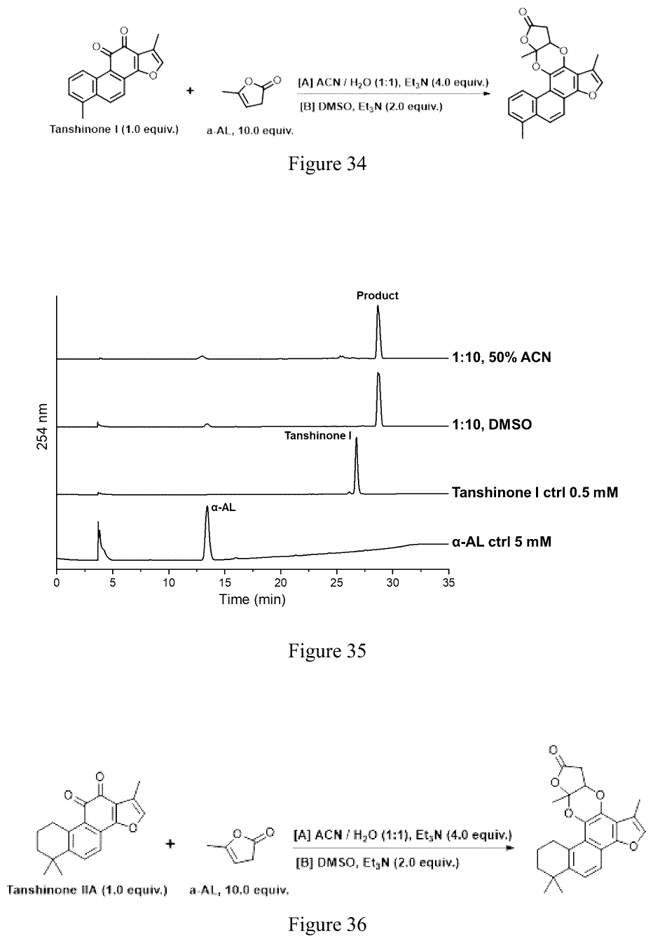

The RNase can be linker with a RNaseL-al molecule. The RNA binder (e.g., miRNA) can be linked with a PQ molecule (PremiR155-PQ, c-Myc-PQ and C-Jun-PQ). The RNaseL-al molecule and the PQ molecule can react with each other for 2 min through a DFC reaction in cell. The ration of RNaseL-al molecule and the PQ molecule may be 1:1. The synthesis route of RNaseL-al molecule is shown in FIG. 33. The HPLC results of the DFC reaction were shown in FIG. 34. The results shows that DFC reaction can be used to connect RNAase and miRNA.

### Example 7

### DFC-Based Bioconjugation to Prepare Tanshinone Derivative

### Tanshinone I Derivative

0.5 mM Tanshinone I were dissolved in the mixed solvents to give a clear solution. α-AL (10 equiv.) and Et₃N (4.0 equiv.) was added to the solution at room temperature and stirred for 10 min. A small aliquot was taken out and quenched with the mixed solvents of MeOH/H₂O/HCOOH before subjection to HPLC and LC-MS analysis. The synthesis route was shown in FIG. 34 and the results was shown in FIG. 35. Tanshinone I derivative was synthesized successfully.

### Tanshinone IIA Derivative

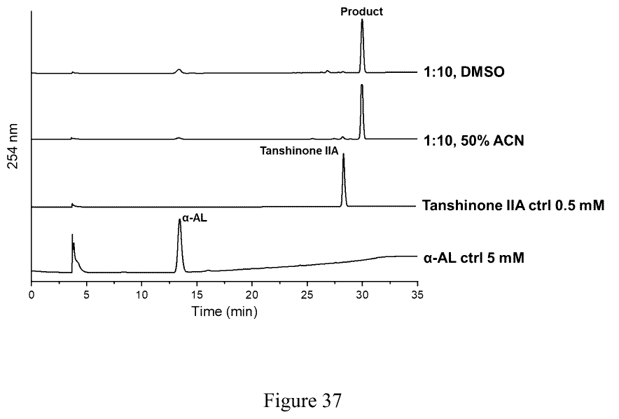

0.5 mM Tanshinone IIA were dissolved in the mixed solvents to give a clear solution. α-AL (10 equiv.) and Et₃N (4.0 equiv.) was added to the solution at room temperature and stirred for 10 min. A small aliquot was taken out and quenched with the mixed solvents of MeOH/H₂O/HCOOH before subjection to HPLC analysis. The synthesis route was shown in FIG. 36 and the results was shown in FIG. 37. Tanshinone IIA derivative was synthesized successfully.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of performing a coupling reaction, comprising providing a first structure comprising a dione group or derivative thereof and a second structure capable of providing anionic furan-2-olate or derivative thereof.

2. The method of claim 1, said second structure comprises a furan-2(3H)-one or derivative thereof.

3. The method of claim 2, said furan-2(3H)-one is optionally substituted; when said furan-2(3H)-one is substituted, said substitution is selected form optional substituents.

4. The method of claim 1, said second structure further comprises small molecule, peptide, nucleic acids, peptide nucleic acid (PNA), oligosaccharides, glycans, and/or lipid.

5. The method of claim 1, said second structure further comprises therapeutic drug, toxin, detectable substance, antibody, fluorescent protein, membrane bound protein and/or glycoprotein.

6. (canceled)

7. The method of claim 1, said first structure comprising an o-dione group or derivative thereof.

8. The method of claim 1, said first structure comprising a phenanthrene-9,10-dione or derivative thereof.

9. (canceled)

10. The method of claim 1, said first structure further comprises small molecule, peptide, nucleic acids, peptide nucleic acid (PNA), oligosaccharides, glycans, and/or lipid.

11. The method of claim 1, said first structure further comprises therapeutic drug, toxin, detectable substance, antibody, fluorescent protein, membrane bound protein and/or glycoprotein.

12. (canceled)

13. The method of claim 1, said method further comprises adding said dione group to said first structure and/or adding said group capable of providing anionic furan-2-olate or derivative thereof to said second structure.

14. (canceled)

15. (canceled)

16. (canceled)

17. (canceled)

18. (canceled)

19. (canceled)

20. A conjugate, said conjugate is prepared using method of claim 1.

21. (canceled)

22. A method for performing two, three or more bioorthogonal reactions, wherein one of the bioorthogonal reaction comprises dione-based cycloaddition reaction, wherein said dione-based cycloaddition reaction comprises method of claim 1.

23. (canceled)

24. (canceled)

25. The method of claim 22, wherein two, three or more of said bioorthogonal reactions are performed simultaneously for mutually orthogonal labelling of three types of proteins or three groups of living cells.

26. (canceled)

27. (canceled)

28. The method of claim 22, wherein one of the bioorthogonal reaction comprises Cu-catalyzed azide-alkyne click reaction (CuAAC), the strain-promoted azide-alkyne click reaction (SPAAC) and/or the inverse-electron-demand Diels-Alder reaction (IEDDA).

29. (canceled)

30. (canceled)

31. (canceled)

32. A method for preparing (targeting chimera) TAC-type molecule, said method comprises providing dione-based cycloaddition reaction, wherein said dione-based cycloaddition reaction comprises method of claim 1.

33. (canceled)

34. The method of claim 32, said TAC-type molecule comprises proteolysis targeting chimera (PROTAC), autophage targeting chimera (AUTAC), lysosome-targeting chimera (LYTAC), bispecific aptamer chimera, antibody-based PROTAC (AbTAC), covalent nanobody-based PROTAC (GlueTAC) and/or ribonuclease targeting chimera (RIBOTAC).

35. (canceled)

36. (canceled)

37. The method of claim 32, said method is performed in vivo, in vitro and/or in situ in living cells.

38. The method of claim 35, said ligand A and said ligand B is linked before or after that said ligand A and/or said ligand B is introduced into living cell.

39. (canceled)

40. A TAC-type molecule prepared using method of claim 32.

41. A method for preparing bi-functional bioconjugate and/or cyclic peptide, said method comprises providing dione-based cycloaddition reaction, wherein said dione-based cycloaddition reaction comprises method of claim 1.

42. (canceled)

43. (canceled)

44. (canceled)

45. (canceled)

46. (canceled)

47. (canceled)

48. (canceled)


SEQUENCE LISTING (1 sequence)

SEQ ID NO: 1 - protein, 440 aa, synthetic construct
HHHHHHGSGGGMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGPARSMGKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKGHRSC